(19) United States
(12) Patent Application Publication
(10) Pub. No.: US 2023/0390349 A1
(43) Pub. Date: Dec. 7, 2023

(54) FUSOBACTERIUM BACTERIOPHAGE AND USES THEREOF

(71) Applicant: Biomx Ltd., Nes Ziona (IL)

(72) Inventors: Lior Moshe ZELCBUCH, Herzeliya (IL); Sagit YAHAV, Kiryat Gat (IL); Maya KAHAN-HANUM, Hod Hasharon (IL); Naomi Bluma ZAK, Jerusalem (IL); Lihi NINIO-MANY, Tel-Aviv (IL)

(73) Assignee: Biomx Ltd., Nes Ziona (IL)

(21) Appl. No.: 18/033,822

(22) PCT Filed: Nov. 11, 2021
(86) PCT No.: PCT/IL2021/051350

§ 371 Date: Apr. 26, 2023

Related U.S. Application Data
(60) Provisional application No. 63/112,729, filed on Nov. 12, 2020.
(60) Provisional application No. 63/183,698, filed on May 4, 2021.

(51) Int. Cl. A61K 35/76; A61P 35/00

(52) U.S. Cl. CPC A61K 35/76; A61P 35/00

(57) ABSTRACT

A method of treating a disease associated with Fusobacterium nucleatum infection in a subject in need thereof is disclosed. The method comprises administering to the subject a therapeutically effective amount of at least one isolated bacteriophage capable of infecting a Fusobacterium nucleatum bacterial species, wherein said at least one bacteriophage has a genomic nucleic acid sequence at least 85% identical to the nucleic acid sequence as set forth in SEQ ID NOs: 1-7 or 15-20. Compositions for treating the disease are also disclosed.

### RELATED APPLICATIONS

This application claims the benefit of priority of U.S. Provisional Patent Application No. 63/112,729 filed 12 Nov. 2020 and U.S. Provisional Patent Application No. 63/183,698, filed 4 May 2021, the contents of which are incorporated herein by reference in their entirety.

### SEQUENCE LISTING STATEMENT

The ASCII file, entitled 89717SequenceListing.txt, created on 9 Nov. 2021, comprising 686,714 bytes, submitted concurrently with the filing of this application is incorporated herein by reference.

### FIELD AND BACKGROUND OF THE INVENTION

The present invention, in some embodiments thereof, relates to bacteriophage capable of infecting Fusobacterium nucleatum bacteria and uses thereof.

Fusobacterium nucleatum (FN) is a Gram-negative, anaerobic bacteria that is part of the oral microflora and considered to be a periodontal pathogen (B. Signat et al., Curr Issues Mol Biol., 2011; 13(2):25-36). Many recent studies have demonstrated associations between the presence of this bacterial species and a cancerous phenotype. FN was demonstrated to be significantly enriched in human colorectal adenomas and carcinomas compared with matched adjacent noncancerous tissue or tissue from healthy controls (H. Hussan et al., World J Gastroenterol., 2017 Dec. 28; 23(48):8626-86, 2017; A. D. Kostic et al., Appl Environ Microbiol., 2010 November; 76(21):7243-50). FN DNA is also overabundant in pancreas (K. Mitsuhashi et al., Oncotarget, 2015 Sep. 8; 6(26):22114-25), breast (T. J. Hieken et al., Sci Rep., 2016 Aug. 3; 6:30751), and esophageal (K. Yamamura et al., Clin Cancer Res., 2016 Nov. 15; 22(22):5574-5581) adenocarcinomas as well as in gastric cancer samples (J. Dicksved et al., J Med Microbiol., 2009 April; 58(Pt 4):509-16; G. Nardone et al., United European Gastroenterol J., 2015 June; 3(3):255-60). Fusobacteria is abundant in oral squamous cell carcinoma and predominant in late stage oral tumors (Z. Zhang et al., Front Microbiol. 2019 Jun. 26; 10:1439). FN has been reported as a dominant species in bile of gall bladder patients (Y. Tsuchiya et al., Asian Pacific J of Cancer Prev. 2018 Apr. 25; 19 (4): 961-967) and to be more abundant in urine from bladder cancer patients (V. Bucevic et al., Sci. Rep. 2018 Aug. 14:8(1):12157). Higher representation of Fusobacterium has been reported in the fecal material of lung cancer patients (W Q Zhang et al., Am. J. Transl. Res. 2018 Oct. 15: 10(10): 3171-3185) and melanoma skin samples of a melanoma pig model (J. Mrazek et al., (Folia Microb. (Praha) 2019 May; 64(30:435-442). Higher abundance of FN has been associated with advanced tumor stage and poor prognosis in colorectal cancer (K. Mima et al., Gut, 2016 December; 65(12):1973-1980). FN may also confer chemoresistance to tumors; FN was found to be abundant in colorectal cancer tissues of patients with recurrent disease post chemotherapy (T. Yu et al., Cell, 2017 Jul. 27; 170(3):548-563).

FN is classified into several subspecies that include nucleatum, animalis, vincentii and polymorphum. All 4 of these subspecies were identified in colorectal adenomas and adenocarcinomas by carrying out qPCR on DNA extracted from these tissues using Fusobacterium specific primers and then subsequently sequencing the amplicons obtained to determine the presence of individual Fusobacterium species and subspecies (X. Ye et al., Cancer Prev Res (Phila)., 2017 July; 10(7):398-409).

There are four reports of bacteriophage that infect or have been extracted from FN. One (P. Machuca et al., App Env Microbiol, 2010 November; 76(21):7243-50) describes the isolation of a Fusobacterium specific phage from the saliva of a healthy individual. The isolated phage efficiently formed plaques on bacterial lawns of Fusobacterium nucleatum polymorphum strains, but isolates of FN subsp. nucleatum and vincentii demonstrated low sensitivity to infection by plaque assay when 5 ul of the phage were applied at a concentration of 10⁷ pfu/ml. Only a small fragment of less than 400 bp of the phage DNA was sequenced and reported to show 93% identity to a P. acnes phage. In a second report (K. Cochrane et al., Anaerobe, 2016 April; 38:125-129), two temperate bacteriophage were isolated from the lysates of a Fusobacterium nucleatum animalis bacterial host that had been treated with mitomycin to induce prophage activation and excision from the host DNA. However, no infection of any host FN strain by these phage was observed (in either their original animalis host or in representatives of the animalis, vincentii and polymorphum subspecies). Thus, these phage were unable to be propagated. The authors suggest that the inability of these phage to infect a host could have been due to either the absence of any putative tail protein or endolysin encoding genes, or a defective tail structure. The third report (M. Kabwe et al., Sci Rep., 2019 Jun. 24; 9(1):9107) describes a lytic DNA phage isolated on Fusobacterium nucleatum polymorphum. The fourth report (D-W. Zheng et al., Nat. Biomed. Eng. 2019 July doi: 10.1038/s41551-019-0423-2) describes a DNA phage that infects subspecies polymorphum, nucleatum and vincentii, but provides no sequence information.

Given the high prevalence and abundance of FN species in tumors and the correlation of the presence of this bacteria with disease prognosis, FN elimination is a promising new modality for treatment of cancer, including oral and esophageal cancer, colorectal cancer, pancreatic cancer, gastric cancer, gall bladder cancer, breast cancer, lung cancer, bladder cancer and melanoma. In addition, highly targeted delivery of anti-cancer payloads to tumors using agents directed against the FN species offers a novel approach for localized cancer treatment.

### SUMMARY OF THE INVENTION

According to an aspect of the present invention there is provided a method of treating a disease associated with a Fusobacterium nucleatum infection in a subject in need thereof, comprising administering to the subject a therapeutically effective amount of at least one isolated bacteriophage capable of infecting a Fusobacterium nucleatum bacterial species causing the infection, wherein the at least one bacteriophage has a genomic nucleic acid sequence at least 85% identical to the nucleic acid sequence as set forth in SEQ ID NOs: 1, 2, 3, 4, 5, 6, 7, 15, 16, 17, 18, 19 or 20, thereby treating the disease associated with the Fusobacterium nucleatum infection.

According to an aspect of the present invention there is provided a method of treating a disease associated with a Fusobacterium nucleatum infection in a subject in need thereof, comprising administering to the subject a therapeutically effective amount of at least one isolated bacteriophage capable of infecting a Fusobacterium nucleatum bacterial species causing the infection, wherein the bacteriophage has a genomic nucleic acid sequence at least 85% identical to the genomic nucleic acid sequence of a bacteriophage deposited with the DSMZ under deposit number DSM 33306, DSM 33307, DSM 33582, DSM 33583, DSM 33639, DSM 33640, DSM 33641, DSM 33642 or DSM 33644, thereby treating the disease associated with the Fusobacterium nucleatum infection.

According to an embodiment of the present invention, the disease is cancer.

According to an embodiment of the present invention, the disease is a periodontal disease or an inflammatory bowel disease.

According to an embodiment of the present invention, the cancer is selected from the group consisting of oral and esophageal cancer, colorectal cancer, pancreatic cancer, gastric cancer, gall bladder cancer, breast cancer, lung cancer, bladder cancer and melanoma.

According to an embodiment of the present invention, the administering comprises intratumoral administration.

According to an embodiment of the present invention, the method further comprises identifying at least one strain of Fusobacterium nucleatum colonizing the subject prior to the administering.

According to an embodiment of the present invention, the at least one bacteriophage is deposited with the DSMZ under deposit number DSM 33582 or DSM 33583.

According to an embodiment of the present invention, the at least one bacteriophage has a genomic nucleic acid sequence at least 85% identical to the nucleic acid sequence as set forth in SEQ ID NOs: 3, 4 or 5.

According to an embodiment of the present invention, the method comprises administering to the subject at least two bacteriophages, wherein each of the at least two bacteriophages is capable of infecting a non-identical Fusobacterium nucleatum bacterial sub-species.

According to an embodiment of the present invention, the method comprises administering to the subject two or more different bacteriophages, wherein the two or more different bacteriophages are capable of infecting different Fusobacterium nucleatum bacterial strains.

According to an embodiment of the present invention, the method comprises administering to the subject at least two bacteriophages, wherein each of the at least two bacteriophages is capable of infecting an identical Fusobacterium nucleatum bacterial sub-species.

According to an embodiment of the present invention, the method comprises administering to the subject two or more different bacteriophages, wherein at least two of the two or more bacteriophages are capable of infecting an identical Fusobacterium nucleatum bacterial sub-species.

According to an embodiment of the present invention, the at least one bacteriophage is genetically modified such that the genome thereof comprises a heterologous sequence.

According to an embodiment of the present invention, the heterologous sequence encodes a therapeutic agent or a diagnostic agent.

According to an embodiment of the present invention, the bacteriophage has a genomic nucleic acid sequence at least 85% identical to the nucleic acid sequence as set forth in SEQ ID NOs: 1, 2, 6 or 7, or a genomic nucleic acid sequence at least 85% identical to the genomic nucleic acid sequence of a bacteriophage deposited with the DSMZ under deposit number DSM 33306, DSM 33307, DSM 33639 or DSM 33644, and wherein the heterologous sequence renders the bacteriophage lytic.

According to an embodiment of the present invention, the therapeutic or diagnostic agent is attached to the outer surface of the bacteriophage.

According to an embodiment of the present invention, the therapeutic agent comprises an immune modulating agent.

According to an aspect of the present invention there is provided a recombinant bacteriophage capable of infecting a Fusobacterium nucleatum bacterial species, wherein the bacteriophage has a genomic nucleic acid sequence at least 85% identical to the nucleic acid sequence as set forth in SEQ ID NOs: 1, 2, 3, 4, 5, 6, 7, 15, 16, 17, 18, 19 or 20, and wherein the bacteriophage is genetically modified such that the genome thereof comprises a heterologous sequence.

According to an aspect of the present invention there is provided a recombinant bacteriophage capable of infecting a Fusobacterium nucleatum bacterial species, wherein the bacteriophage has a genomic nucleic acid sequence at least 85% identical to the genomic nucleic acid sequence of a bacteriophage deposited with the DSMZ under deposit number DSM 33582, DSM 33583, DSM 33306, DSM 33639, DSM 33640, DSM 33641, DSM 33642 or DSM 33644, or DSM 33307, and wherein the bacteriophage is genetically modified such that the genome thereof comprises a heterologous sequence.

According to an embodiment of the present invention, the heterologous sequence encodes a therapeutic agent or a diagnostic agent.

According to an embodiment of the present invention, the bacteriophage has a genomic nucleic acid sequence at least 85% identical to the nucleic acid sequence as set forth in SEQ ID NOs: 1, 2, 6 or 7, and the heterologous sequence renders the bacteriophage lytic.

According to an embodiment of the present invention, the bacteriophage has a genomic nucleic acid sequence at least 85% identical to the genomic nucleic acid sequence of a bacteriophage deposited with the DSMZ under deposit number DSM 33306, DSM 33307, DSM 33639 or DSM 33644, the heterologous sequence renders the bacteriophage lytic.

According to an aspect of the present invention there is provided a composition comprising two or more different isolated bacteriophages capable of infecting a Fusobacterium nucleatum bacterial strain, wherein at least one of the two or more different bacteriophages has a genomic nucleic acid sequence at least 85% identical to the nucleic acid sequence as set forth in SEQ ID NOs: 1, 2, 3, 4, 5, 6, 7, 15, 16, 17, 18, 19 or 20, or has a genomic nucleic acid sequence at least 85% identical to the genomic nucleic acid sequence of a bacteriophage deposited with the DSMZ under deposit number DSM 33306, DSM 33582, DSM 33307, DSM 33583, DSM 33639, DSM 33640, DSM 33641, DSM 33642 or DSM 33644.

According to an embodiment of the present invention, two of two or more isolated bacteriophages has a genomic nucleic acid sequence at least 85% identical to the nucleic acid sequence as set forth in SEQ ID NOs: 1, 2, 3, 4, 5, 6, 71, 5, 16, 17, 18, 19 or 20, or has a genomic nucleic acid sequence at least 85% identical to the genomic nucleic acid sequence of a bacteriophage deposited with the DSMZ under deposit number DSM 33306, DSM 33582, DSM 33307, DSM 33583, DSM 33639, DSM 33640, DSM 33641, DSM 33642 or DSM 33644.

According to an embodiment of the present invention, each of the two or more bacteriophages is capable of infecting a non-identical Fusobacterium nucleatum bacterial sub-species.

According to an embodiment of the present invention, each of the at least two bacteriophages is capable of infecting an identical Fusobacterium nucleatum bacterial sub-species.

According to an embodiment of the present invention, at least two of the two or more bacteriophages is capable of infecting a non-identical Fusobacterium nucleatum bacterial sub-species.

According to an embodiment of the present invention, at least two of the two or more bacteriophages are capable of infecting an identical Fusobacterium nucleatum bacterial sub-species.

According to an embodiment of the present invention, the at least one bacteriophage is genetically modified such that the genome thereof comprises a heterologous sequence.

According to an embodiment of the present invention, the at least one bacteriophage is attached to a therapeutic or diagnostic agent.

According to an embodiment of the present invention, the heterologous sequence encodes a therapeutic agent or a diagnostic agent.

According to an embodiment of the present invention, the at least one bacteriophage has a genomic nucleic acid sequence at least 85% identical to the nucleic acid sequence as set forth in SEQ ID NOs: 1, 2, 6 or 7, or has a genomic nucleic acid sequence at least 85% identical to the genomic nucleic acid sequence of a bacteriophage deposited with the DSMZ under deposit number DSM 33306, DSM 33307, DSM 33639 or DSM 33644, and the heterologous sequence renders the at least one bacteriophage lytic.

According to an embodiment of the present invention, the therapeutic agent comprises an immune modulating agent.

According to an aspect of the present invention there is provided a pharmaceutical composition comprising a recombinant bacteriophage described herein as the active agent, and a pharmaceutical carrier.

According to an embodiment of the present invention, the pharmaceutical composition is formulated for intratumoral delivery.

According to an embodiment of the present invention, the composition is formulated for intratumoral delivery.

According to an aspect of the present invention there is provided an isolated bacteriophage capable of infecting a Fusobacterium nucleatum bacterial species, wherein the bacteriophage has a genomic nucleic acid sequence at least 85% identical to the nucleic acid sequence as set forth in SEQ ID NOs: 1, 2, 3, 4, 5, 6, 7 15, 16, 17, 18, 19 or 20, or having a genomic nucleic acid sequence at least 85% identical to the genomic nucleic acid sequence of a bacteriophage deposited with the DSMZ under deposit number DSM 33307, DSM 33306, DSM 33582, DSM 33583, DSM 33639, DSM 33640, DSM 33641, DSM 33642 or DSM 33644.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 depicts the comparative growth curves of FN1 bacteria alone, or in the presence of different multiplicity of infection (MOI) of phage FN1-1, as measured by optical density.

FIGS. 2A-C depict the incorporation of F. nucleatum (FN5) into subcutaneous murine colon carcinoma tumors as shown by measurement of CFUs recovered per gram tumor 2A) and per whole tumor (2B). The effect of different doses of FN5 on tumor growth is shown in 2C.

FIG. 3A depicts the schedule for tumor, FN5 bacteria, and FN5-1 phage inoculation into mice.

FIG. 3B depicts the genome of a lysogenized F. nucleatum. The location of the prophage and PCR primers and probe are marked.

FIG. 3C depicts a dot plot showing the amount of lysogenized FN5 in the presence or absence of FN5-1 phage administration.

FIG. 4 depicts the effect of a wild type lysogenic lambda phage (wt), a synthetic, repressor-less version of that wild-type lambda phage (Δcl), and a known lambda lytic phage mutant Vir (vir) on E. coli growth.

FIG. 5 depicts a shuttle plasmid used to insert a payload into the genome of a temperate phage specific for an FN host using homologous recombination, which also inactivates the integrase gene.

FIG. 6A is a photograph of a Western blot showing the expression of HiBiT-tagged mouse IL-15. WT-wild type phage; IL15-phage genome contains IL-15,FNP-phage genome contains native FN protein (enolase).

FIG. 6B is a photograph of a Western blot showing the expression of HiBiT-tagged pyruvate kinase and enolase.

FIG. 6C depicts the desired area of deletion in an exemplary shuttle plasmid as illustrated in FIG. 5.

FIGS. 7A-B depict how a payload plasmid can be transferred into a clinical F. nucleatum strain.

FIGS. 8A-B depict recombinant FN14-1 phage expressing an HiBiT-tagged-Integrase. The integrase of FN-14-1 phage was tagged with a C-terminal HiBiT tag using a homologous recombination method. The recombinant phage (expressing an HiBiT-tagged integrase) was detected by PCR (using a selective primers pair for the recombinant phage, FIG. 8A) and by Luciferase assay (FIG. 8B).

FIGS. 8C-E depict results of an experiment demonstrating that IL-15 engineered phage can successfully infect fusobacterium in a mouse cancer model. FIG. 8C illustrates fusobacterium colonization in tumors. FIG. 8D illustrates the amount of IL-15 engineered phage in phage-positive tumors. FIG. 8E illustrates detection of IL-15 payload, 4 days post phage administration.

FIGS. 9A-C illustrates F. nucleatum phage engineered from lysogenic to lytic. FIG. 9A is a diagram illustrating how the phage was engineered. FIG. 9B is a photograph of wild-type phage. FIG. 9C is a photograph of the engineered phage.

FIG. 10 is a graph illustrating that natural phage accumulate in bacteria colonized tumors. PFU assay on tumors extracted from mice inoculated with bacteria and treated with vehicle (FN+vehicle), treated with phages (FN+Phages) or treated with phages without bacteria colonization (Phage only) **p<0.05.

FIG. 11 is a bar graph illustrating a significant decrease in F. nucleatum load following natural phage treatment. qPCR of bacterial load in tumors inoculated with bacteria treated with vehicle control (FN) or treated with phages (FN+phages), **p<0.05. (n=4, each group).

FIG. 12 is a graph illustrating a reduction in tumor volume in groups treated with phage compared to vehicle control group.

### DESCRIPTION OF SPECIFIC EMBODIMENTS OF THE INVENTION

The present invention, in some embodiments thereof, relates to bacteriophage capable of infecting Fusobacterium nucleatum bacteria and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have isolated bacteriophage characterized by having a high specificity to one or more particular FN subspecies (see Table 1) and having a high level of intrinsic safety, as they do not recognize human cells. For elimination of pathogenic FN subspecies, the present disclosure provides lytic bacteriophage, which do not have any capacity to integrate into the DNA of their bacterial host. Such bacteriophage bring about immediate target bacterial eradication through lysis after hijacking the host protein expression machinery to manufacture needed phage protein components. For delivery of therapeutic agents, the present invention provides both lytic phage and lysogenic phage that integrate into the DNA of an FN bacterial host, are replicated within that bacteria, and whose DNA payload may direct continued expression by the FN bacteria of the therapeutic agent.

Genetic manipulation of Fusobacterium nucleatum phage inside their host is difficult, due to the anaerobic growth conditions, low genomic GC content and multiple restriction systems of Fusobacterium nucleatum. (S. Coppenhagen-Glazer S et al., Infect Immun., 2015 March; 83(3):1104-13).

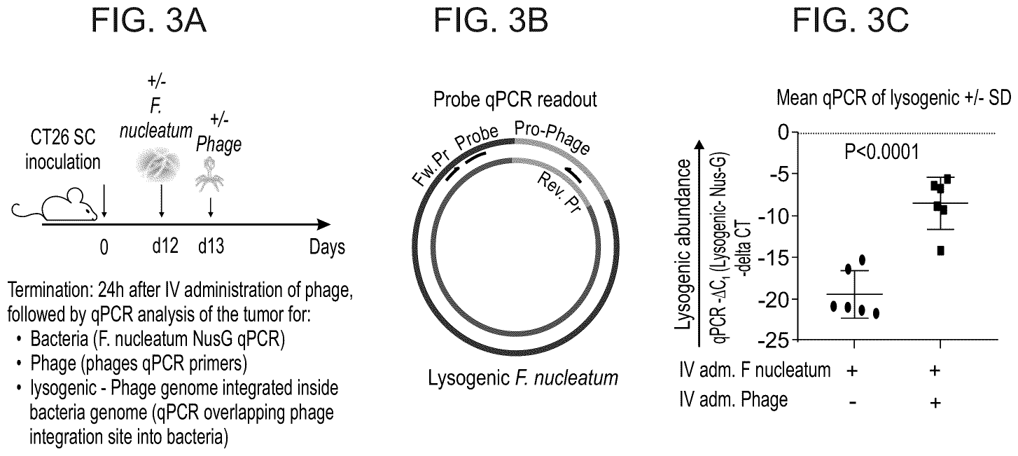

Whilst reducing the present invention to practice, the present inventors have demonstrated homing of FN phage to an FN bearing tumor following intravenous administration (FIGS. 3A-C). Using an animal model of a tumor with embedded FN, the present inventors have intravenously administered a lysogenic phage which may be inserted into the FN DNA in the tumor. This was observed by designing a PCR assay which specifically amplifies a PCR product with one primer recognizing sequences inside the phage and a second primer that recognizes the host DNA adjacent to the site of prophage insertion. This demonstrates the feasibility of employing phage of the present disclosure to target FN bacteria found inside tumors.

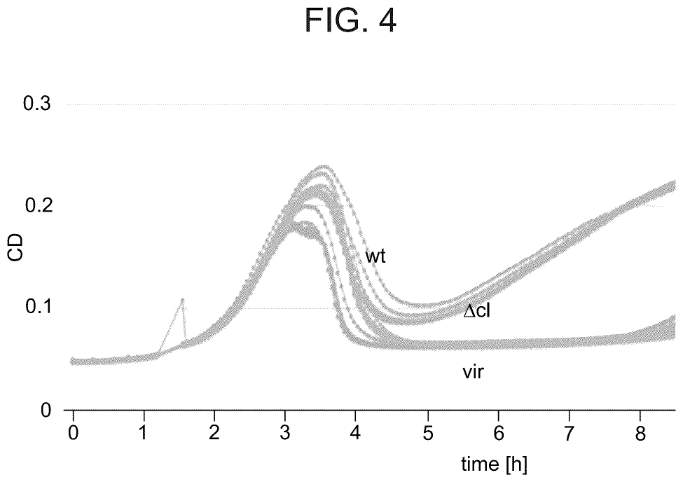

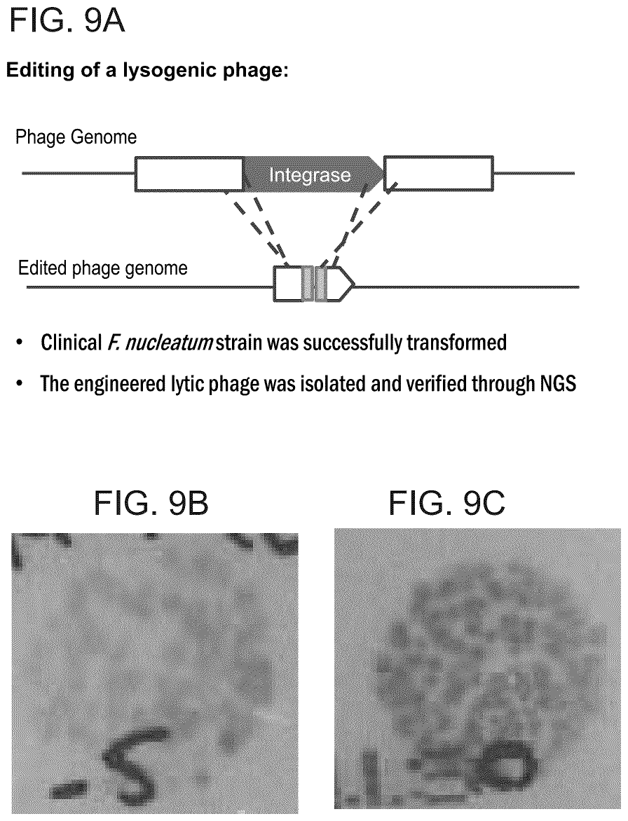

The present inventors have also shown that it is possible to convert a lysogenic phage into a lytic phage (FIG. 4 and FIGS. 9A-C). Engineering of such phage further enlarges the panel of lytic phage against FN that can be used to eradicate FN bacteria responsible for the disease.

As is illustrated herein under and in the examples section which follows, the present inventors show that it is possible to engineer FN phage to express a heterologous sequence (Table 5) pathing the way for the development of recombinant FN phage which express a therapeutic agent.

Thus, according to a first aspect of the present invention, there is provided an isolated bacteriophage capable of infecting a Fusobacterium nucleatum bacterial species, wherein the bacteriophage has a genomic nucleic acid sequence at least 85% identical to the nucleic acid sequence as set forth in SEQ ID NOs: 1, 2, 3, 4, 5, 6, 7, 15, 16, 17, 18, 19 or 20 or having a genomic nucleic acid sequence at least 85% identical to the genomic nucleic acid sequence of a bacteriophage deposited with the DSMZ under deposit number DSM 33307, DSM 33306, DSM 33582 or DSM 33583.

As used herein, the term “bacteriophage” and “phage” are used interchangeably and refer to an isolated virus that is capable of infecting a bacterium. Typically, a phage will be characterized by: 1) the nature of the nucleic acids that make up its genome, e.g., DNA, RNA, single-stranded or double-stranded; 2) the nature of its infectivity, e.g., lytic or temperate; and 3) the particular FN subspecies that it infects (and in certain instances the particular strain of that FN subspecies). This aspect is known as “host range”.

As used herein, the phrase “isolated bacteriophage”, “isolate” or grammatical equivalents as used herein refers to a bacteriophage which is removed from its natural environment (e.g. removed from bacteria which it typically infects). In one embodiment, the isolated bacteriophage is removed from cellular material and/or other elements that naturally exist in the source clinical or environmental sample. The term isolated bacteriophages includes such phages isolated from human or animal subjects (“clinical isolates” or “clinical variants”) and such phages isolated from the environment (“environmental isolates”).

As used herein, the phrase “phage strain” refers to the deposited or sequenced phage, as described herein.

The terms, “Fusobacteria nucleatum” and “FN” are used interchangeably and relate to a specific species of the anaerobic, gram negative Fusobacterium genus. There are four known subspecies of Fusobacteria nucleatum-Fusobacteria nucleatum nucleatum, Fusobacteria nucleatum animalis, Fusobacteria nucleatum vincentii, and Fusobacteria nucleatum polymorphum.

Also contemplated are functional homologs of those that are deposited with the DSMZ under deposit number DSM 33307, DSM 33306, DSM 33582, DSM 33583, DSM 33639, DSM 33640, DSM 33641, DSM 33642 or DSM 33644, wherein the functionally homologous bacteriophage is capable of infecting the same strain of Fusobacterium nucleatum (or even subspecies of Fusobacterium nucleatum) as that which the deposited bacteriophage infects.

Also contemplated are functional homologs of those that have a genomic nucleic acid sequence as set forth in SEQ ID NOs: 1, 2, 3, 4, 5, 6, 7, 15, 16, 17, 18, 19 or 20, wherein the functionally homologous bacteriophage is capable of infecting the same strain (or even subspecies) of Fusobacterium nucleatum as that which the bacteriophage having one of the above set forth genomic nucleic acid sequence infects.

As used herein “functional homolog” or “functionally homologous” or “variant” or grammatical equivalents as used herein refers to a bacteriophage with a genomic nucleic acid sequence different than that of the deposited bacteriophage (i.e., at least one mutation) resulting in a bacteriophage that is endowed with substantially the same ensemble of biological activities (+/−10%, 20%, 40%, 50%, 60% when tested under the same conditions) as that of the deposited bacteriophage and can be classified as infecting the same subspecies or strain of bacteria based on known methods of species/strain classifications.

A bacteriophage “infects” bacteria if it either causes the bacteria to lyse or integrates its nucleic acid sequence into the bacterial genome.

According to a specific embodiment, the bacteriophage has a genomic nucleic acid sequence at least 85% homologous (% identical) to the nucleic acid sequence as set forth in SEQ ID NOs: 1, 2, 3, 4, 5, 6 or 7.

According to another embodiment, the genomic nucleic acid sequence of the bacteriophage comprises sequence at least 85% homologous (% identical) to the nucleic acid sequence as set forth in SEQ ID NOs: 15-17.

According to another embodiment, the genomic nucleic acid sequence of the bacteriophage comprises sequence at least 85% homologous (% identical) to the nucleic acid sequence as set forth in SEQ ID NOs: 18-20.

As used herein, “percent homology”, “percent identity”, “sequence identity” or “identity” or grammatical equivalents as used herein in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

It will be appreciated that the percent identity of the genomic nucleic acid sequence of the bacteriophage is not calculated to include heterologous nucleic acid sequences that have been incorporated into anyone of the aligned genomes of the phage. Thus, the percent identity of the bacteriophage genomic nucleic acid sequence excludes heterologous nucleic acid sequences that encode payloads such as therapeutic agents or diagnostic agents. In addition, the percent identity of the genomic nucleic acid sequence of the bacteriophage is not calculated to include targeted deletions such as those used to convert the phage from a temperate phage to a lytic phage, as further described below.

Percent identity can be determined using any homology comparison software, including for example, the BlastN software of the National Center of Biotechnology Information (NCBI) such as by using default parameters.

Other exemplary sequence alignment programs that may be used to determine % homology or identity between two sequences include, but are not limited to, the FASTA package (including rigorous (SSEARCH, LALIGN, GGSEARCH and GLSEARCH) and heuristic (FASTA, FASTX/Y, TFASTX/Y and FASTS/M/F) algorithms, the EMBOSS package (Needle, stretcher, water and matcher), the BLAST programs (including, but not limited to BLASTN, BLASTX, TBLASTX, BLASTP, TBLASTN), megablast and BLAT. In some embodiments, the sequence alignment program is BLASTN. For example, 95% homology refers to 95% sequence identity determined by BLASTN, by combining all non-overlapping alignment segments (BLAST HSPs), summing their numbers of identical matches and dividing this sum with the length of the shorter sequence.

In some embodiments, the sequence alignment program is a basic local alignment program, e.g., BLAST. In some embodiments, the sequence alignment program is a pairwise global alignment program. In some embodiments, the pairwise global alignment program is used for protein-protein alignments. In some embodiments, the pairwise global alignment program is Needle. In some embodiments, the sequence alignment program is a multiple alignment program. In some embodiments, the multiple alignment program is MAFFT. In some embodiments, the sequence alignment program is a whole genome alignment program. In some embodiments, the whole genome alignment is performed using BLASTN. In some embodiments, BLASTN is utilized without any changes to the default parameters.

According to some embodiments of the invention, the identity is a global identity, i.e., an identity over the entire nucleic acid sequences of the invention and not over portions thereof.

According to a specific embodiment, the genomic nucleic acid sequence is at least about 85%, at least about 90%, at least about 91%, at least about 92%, at least about 93%, at least about 94%, at least about 95%, at least about 96% least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9%, at least about 99.95% 99.95%, at least about 99.99%, or more identical to the genomic sequence of the deposited bacteriophage or to one of the genomic sequences as set forth in SEQ ID NOs: 1, 2, 3, 4, 5, 6, 7, 15-17 or 18-20.

According to a specific embodiment, the genomic nucleic acid sequence is at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.8%, at least about 99.9% or more identical to that of the deposited bacteriophage or to one of the genomic sequences as set forth in SEQ ID NOs: 1, 2, 3, 4, 5, 6, 7, 15-17 or 18-20.

According to an additional or alternative embodiment, a functional homolog is determined as the average nucleotide identity (ANI), which detects the DNA conservation of the core genome (Konstantinidis K and Tiedje J M, 2005, Proc. Natl. Acad. Sci. USA 102: 2567-2592). In some embodiments, the ANI between the functional homolog and the deposited bacteriophage (or that having a genome as set forth in any one of SEQ ID NO: 1, 2, 3, 4, 5, 6, 7, 15-17 or 18-20) is of at least about 95%, at least about, 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, a functional homolog is determined by the degree of relatedness between the functional homolog and the deposited bacteriophage (or that having a genome as set forth in any one of SEQ ID NO: 1, 2, 3, 4, 5, 6 or 7) determined as the Tetranucleotide Signature Frequency Correlation Coefficient, which is based on oligonucleotide frequencies (Bohlin J. et al. 2008, BMC Genomics, 9:104). In some embodiments, the Tetranucleotide Signature Frequency Correlation coefficient between the variant and the deposited bacteriophage (or that having a genome as set forth in any one of SEQ ID NO: 1, 2, 3, 4, 5, 6, 7, 15-17 or 18-20) is of about 0.99, 0.999 or more.

According to an additional or alternative embodiment, the degree of relatedness between the functional homolog and the deposited bacteriophage (or that having a genome as set forth in any one of SEQ ID NO: 1, 2, 3, 4, 5, 6 or 7) is determined as the degree of similarity obtained when analyzing the genomes of the parent and of the variant bacteriophage by Pulsed-field gel electrophoresis (PFGE) using one or more restriction endonucleases. The degree of similarity obtained by PFGE can be measured by the Dice similarity coefficient. In some embodiments, the Dice similarity coefficient between the variant and the deposited bacteriophage (or that having a genome as set forth in any one of SEQ ID NO: 1, 2, 3, 4, 5, 6, 7, 15-17 or 18-20) is of at least about 96%, at least about 97%, at least about 98%, at least about 99%, at least about 99.1%, at least about 99.5%, at least about 99.6%, at least about 99.7%, at least about 99.8%, at least about 99.9% or more.

According to an additional or alternative embodiment, the degree of relatedness between the functional homolog and the deposited bacteriophage (or that having a genome as set forth in any one of SEQ ID NO: 1, 2, 3, 4, 5, 6, 7, 15-17 or 18-20) is determined by the Pearson correlation coefficient obtained by comparing the genetic profiles of both phages obtained by repetitive extragenic palindromic element-based PCR (REP-PCR) (see e.g. Chou and Wang, Int J Food Microbiol. 2006, 110:135-48). In some embodiments, the Pearson correlation coefficient obtained by comparing the REP-PCR profiles of the variant and the deposited phage is of at least about 0.99, at least about 0.999 or more—see for example bmcmicrobioldotbiomedcentraldotcom/articles/10.1186/s12866-020-01770-2.

According to an additional or alternative embodiment, the degree of relatedness between the functional homolog and the deposited bacteriophage is defined by the linkage distance obtained by comparing the genetic profiles of both phages obtained by Multilocus sequence typing (MLST) (see e.g. Maiden, M. C., 1998, Proc. Natl. Acad. Sci. USA 95:3140-3145). In some embodiments, the linkage distance obtained by MLST of the functional homolog and the deposited phage (or that having a genome as set forth in any one of SEQ ID NO: 1, 2, 3, 4, 5, 6, 7, 15-17 or 18-20) is of at least about 0.99, at least about 0.999 or more.

According to an additional or alternative embodiment, the functional homolog comprises a functionally conserved gene or a fragment thereof e.g., an integrase gene, a polymerase gene, a capsid protein assembly gene, a DNA terminase, a tail fiber gene, or a repressor gene that is at least about 97%, at least about 98%, at least about 99%, at least about 99.1%, at least about 99.5%, at least about 99.6%, at least about 99.7%, at least about 99.8%, at least about 99.9%, or more identical to that of the deposited bacteriophage (or that having a genome as set forth in any one of SEQ ID NO: 1, 2, 3, 4, 5, 6, 7, 15-17 or 18-20).

According to an additional or alternative embodiment, the functional homolog is defined by a comparison of coding sequences (gene) order.

According to an additional or alternative embodiment, the functional homolog is defined by a comparison of order of non-coding sequences.

According to an additional or alternative embodiment, the functional homolog is defined by a comparison of order of coding and non-coding sequences.

According to some embodiments of the invention, the combined coding region of the functional homolog is such that it maintains the original order of the coding regions as within the genomic sequence of the deposited bacteriophage (or that having a genome as set forth in any one of SEQ ID NO: 1, 2, 3, 4, 5, 6, 7, 15-17 or 18-20), yet without the non-coding regions.

For example, in case the genomic sequence has the following coding regions, A, B, C, D, E, F, G, each flanked by non-coding sequences (e.g., regulatory elements, and the like), the combined coding region will include a single nucleic acid sequence having the A+B+C+D+E+F+G coding regions combined together while maintaining the original order of their genome, yet without the non-coding sequences.

According to some embodiments of the invention, the combined non-coding region of the functional homolog is such that it maintains the original order of the non-coding regions as within the genomic sequence of the deposited bacteriophage (or that having a genome as set forth in any one of SEQ ID NO: 1, 2, 3, 4, 5, 6, 7, 15-17 or 18-20), yet without the coding regions as originally present in the original bacteriophage.

According to some embodiments of the invention, the combined non-coding region and coding region (i.e., the genome) of the functional homolog is such that it maintains the original order of the coding and non-coding regions as within the genomic sequence of the bacteriophage deposit (or that having a genome as set forth in any one of SEQ ID NO: 1, 2, 3, 4, 5, 6, 7, 15-17 or 18-20).

As used herein “maintains” relate to at least about 90%, 91%, 92%, 93%, 94%, 95%, 96%, 97%, 98%, 99% or 100% of the coding and/or non-coding regions of the functional homolog compared to the deposited bacteriophage.

According to an additional or alternative embodiment, the functional homolog is defined by a comparison of gene content.

According to a specific embodiment, the functional homolog comprises a combined coding region at least about 90%, at least about 91%, at least about 92%, at least about 93%, at least about 94%, at least about 95%, at least about 96%, at least about 97%, at least about 98%, at least about 99%, or more (e.g., 100%) identical to the combined coding region existing in genome of the deposited bacteriophage (or that having a genome as set forth in any one of SEQ ID NO: 1, 2, 3, 4, 5, 6, 7, 15-17 or 18-20).

As used herein “combined coding region” refers to a nucleic acid sequence including all of the coding regions of the original bacteriophage yet without the non-coding regions of the original bacteriophage.

In one embodiment, the bacteriophages show up to 85% sequence identity with the bacteriophages disclosed herein and share at least one of the following characteristics—similar host range; similar type of infectivity (i.e. lytic or temperate).

In another embodiment, the bacteriophages show up to 85% sequence identity with the bacteriophages disclosed herein and share both of the following characteristics—similar host range; similar type of infectivity.

Additional bioinformatics methods that may be used to determine relatedness between two phage genomes include Nucmer and Minimap, both of which are alignment based tools; Win-zip, Jacard distance and MinHash, each of which are information based tools; and Codon usage similarity, pathway similarity and protein motif similarity.

As used herein, “host range” refers to the bacteria that are susceptible to infection by a particular phage. The host range of a phage may include, but is not limited to, a strain, a subspecies, a species, a genus, or multiple genera of bacteria.

Different phage isolates may be prepared and phenotyped using methods known in the art, e.g., a plaque assay, liquid media assay, solid media assay. In some embodiments, the solid media assays to quantify and isolate phage are based on plaque assays (S. T. Abedon et al., Methods in Molecular Biology 2009 (Clifton, N.J.), 501, 161-74), ranging from efficiency of plating (EOP) (E. Kutter, Methods in Molecular Biology 2009 (Clifton, N.J.), 501, 141-9) to spot testing (P. Hyman et al., Advances in Applied Microbiology (1st ed., Vol. 70, pp. 217-48) 2010. Elsevier Inc.). In some embodiments, the plate format used for the plaque assay can be modified, e.g., from a petri dish to a 48-well plate.

In some embodiments, a double-layer plaque assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 100 μL of a phage-containing sample (or medium only control) and incubated for 15 minutes. 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 16 hours at 37° C. in anaerobic environment until plaques are identified.

In some embodiments, a modified spot drop assay is used to phenotype bacteriophage isolates. For example, a starter culture of 4 mL BHIS may be inoculated with 50-100 colonies from a plate. This culture may be incubated at 37° C. for 16 hours in an anaerobic environment. A volume of 200 μL of this culture may be mixed with 5 mL of BHIS top agar (pre-molten 0.4% agar BHIS supplemented with 1 mM Ca²⁺, Mn²⁺ and Mg²⁺ ions may be added), and the mixture may be poured over a BHIS bottom agar plate (1.5% agar BHIS). The plates may be allowed to gel at room temperature, and then incubated for 30 min at 37 C in anaerobic environment. At this stage, 5 μL of samples containing phage or media only as control may be dropped on the plate, left to absorb, and then may be incubated for 16 hours until plaques are visible for counting.

In some embodiments, a liquid media assay is used to phenotype the bacteriophage. In some embodiments, liquid-based phage infection assays follow the time-course of infection and can provide more than quantitative end-points of infection as compared to the solid-phase plaque assays. In some embodiments, by mixing phage with bacteria in liquid medium, then following the turbidity of the culture over time, one can discern finer differences (e.g., a delay in the time of cell lysis) between how different bacterial strains interact with the phage. In some embodiments, the liquid media assay allows for high-throughput measurements by using 96-well plates and reading optical density in a plate reader.

For example, a bacterial strain may be grown for 16 hours until an OD₆₀₀ of about 1.5-2. This culture may then be diluted using BHIS medium (which may be supplemented with 0.1% g/w L-cysteine) to a starting optical density, typically between 0.03 and 0.05 OD₆₀₀ and kept for grow for 6 additional hours. A volume of 200 μL of culture may then be dispensed into the wells of a Nunclon flat-bottomed 96-well plate. 10 μL of a sample containing phage or 10 μL of medium as control may be added to each well. The wells may be covered with 50 μL of mineral oil to limit evaporation, and a thin sterile optically transparent polyester film may be added to keep the culture sterile and anaerobic. Optical density measurements may be carried out every 15 minutes, e.g., in a Tecan Infinite M200 plate reader connected to a Tecan EVO75 robot. Between measurements, the plate may be incubated while shaking at 37° C., e.g., inside the EVO75 incubator.

In some embodiments, infectivity is determined by the plaque presence in a solid assay only. In some embodiments, infectivity is determined by the plaque presence in a liquid assay only. In some embodiments, infectivity is determined by the plaque presence in both the liquid assay and the solid assay.

The bacteriophages described herein are typically present in a preparation in which their prevalence (i.e., concentration) is enriched over that (i.e. exceeds that) found in nature.

The term “preparation” refers to a composition in which the prevalence of bacteriophage is enriched over that found in nature. Since bacteriophages infect bacterial cells, they may be found in specimens or samples which are rich in bacteria—e.g. environmental samples such as sewage, wastewater and biological samples including feces. According to some embodiments of the invention, the preparation comprises less than 50 microbial species, e.g., bacteria and fungi—e.g. less than 40 bacterial species, less than 30 bacterial species, less than 20 bacterial species, less than 10 bacterial species, less than 5 bacterial species, less than 4 bacterial species, less than 3 bacterial species, less than 2 bacterial species or even devoid completely of bacteria.

According to a particular embodiment, the preparation comprises a single strain of bacteriophage (or a functional homolog thereof), no more than two different bacteriophage strains (or functional homologs thereof), no more than three different bacteriophage strains (or functional homologs thereof), no more than four different bacteriophage strains (or functional homologs thereof), no more than five different bacteriophage strains (or functional homologs thereof), no more than six different bacteriophage strains (or functional homologs thereof), no more than seven different bacteriophage strains (or functional homologs thereof), no more than eight different bacteriophage strains (or functional homologs thereof), no more than nine different bacteriophage strains (or functional homologs thereof), or no more than ten different bacteriophage strains (or functional homologs thereof).

The term “different” in the context of “two different bacteriophage strains” refers to bacteriophages which are genetically distinct from one another.

According to a specific embodiment the preparation comprises at least about 10⁶ PFU, 10⁷ PFU, 10⁸ PFU, 10⁹ PFU, or even 10¹⁰ PFU or more of the deposited bacteriophage or functional homolog of same.

The bacteriophages described herein may be genetically modified such that their genomes include a heterologous sequence (i.e. a sequence that is non-native to the wild-type phage). The heterologous sequence may be of any length, for example at least 20 nucleotides, at least 50 nucleotides, at least 100 nucleotides, at least 500 nucleotides or at least 1000 nucleotides.

In one embodiment, the heterologous sequence serves as a marker signifying whether transformation is successful—e.g. a barcode sequence.

In another embodiment, the heterologous sequence encodes at least one therapeutic or diagnostic agent (also referred to herein as a payload). The therapeutic or diagnostic agent may be a nucleic acid (e.g. RNA silencing agent), a peptide or a protein. The therapeutic agent is typically selected according to the disease which is to be treated. Thus, for example if the bacteriophage is to be used for treating cancer, the therapeutic agent is typically one that is known to be useful for treating cancer.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs. In one embodiment, the RNA silencing agent is capable of inducing RNA interference. In another embodiment, the RNA silencing agent is capable of mediating translational repression. According to an embodiment of the invention, the RNA silencing agent is specific to the target RNA and does not cross inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

Exemplary RNA silencing agents include but are not limited to siRNA, shRNA, miRNA and guide RNA (gRNA).

The therapeutic agent may be a bacterial protein or peptide (e.g., a small bacterial peptide that could act as a vaccine in the subject treated with the bacteriophage), a therapeutic protein or peptide (e.g., a cytokine, e.g., IL-15), a soluble peptide or protein ligand (e.g., a STING agonist or TRAIL), an antibody or an antibody fragment that recognizes a virulent or disease-causing antigen or is useful in an immunotherapy (e.g., a checkpoint inhibitor), an enzyme that when expressed produces a therapeutic useful product (e.g., a bacterial enzyme or metabolic cassette that produces a therapeutically useful bacterial metabolite or other bacterial antigen; a bacterial enzyme that produces LPS or causes cleavage of LPS from the outer membrane of gram negative bacteria), a shared tumor antigen or an enzyme that when expressed produces a shared tumor antigen, an agent which activates the immune system such as a TLR agonist or a RIG agonist, a unique tumor antigen or neoantigen or an enzyme that when expressed produces a unique tumor antigen or neoantigen,

In another embodiment, the therapeutic agent is an agent that is therapeutic in the treatment of cancer. The anti-cancer agent may cause reduced growth, reduced proliferation, and/or killing of the cancer cell. The therapeutic agent may be a drug converting enzyme (e.g. converts a pro-drug into a functional drug such as cytosine deaminase.

Exemplary therapeutic agents include, but are not limited to Purine Nucleoside Phosphorylase, Carboxypeptidase P2, Viral thymidine kinase (TK), thymidine phosphorylase (TP), nitroreductase (NTR), D-amino-acid oxidase (DAAO), xanthine-guanine phosphoribosyl transferase (XGPRT), penicillin-G amidase (PGA), β-lactamase (β-L). multiple-drug activation enzyme (MDAE), β-galactosidase (β-Gal), horseradish peroxidase (HRP), deoxyribonucleotide kinase (DRNK), deoxycytidine kinase (dCK), carboxypeptidase A (CPA), β-glucuronidase (β-Glu), and cytochrome P450 (CYP).

In another embodiment, the therapeutic agent is an anti-angiogenesis factor including but not limited to soluble VEGFR, soluble FGFR, Endostatin, Thrombospondin-1, Angiostatin, Vasostatin, NK4, Matrix metalloproteinases inhibitors (MMIPs/TIMPs), somatostatin receptors (SSTR) and Tumstatin.

The therapeutic agent may also be an agent that increases sensitivity to chemotherapy and/or irradiation. Thus, for example the therapeutic agent may be a radiosensitizer or a radiosensitizing agent that potentiates the toxicity of radiation therapy, including, but not limited to PTX, 17-AAG and Rapamaycin.

Sensitivity to radiation and chemotherapy can be influenced by factors extrinsic to the cancer cell. For example, severely hypoxic cells require 2-3 times the radiation dose as do well-oxygenated cells to achieve similar cell killing. Hence, response to cytotoxic therapy may be improved by modulating the tumor microenvironment (TME). Agents can be blockers/inhibitors for vascular endothelial growth factor (VEGF), a secreted protein that regulates angiogenesis, or hypoxia inducible factor-1 alpha (HIF-1 alpha), a master transcription factor that regulates gene expression in hypoxia.

According to another embodiment, the therapeutic agent is an immune modulating agent.

Examples of immune modulating agents include immunomodulatory cytokines, including but not limited to, IL-12, IL-2, IL-15, IL-7, IL-21, GM-CSF as well as any other cytokines that are capable of further enhancing immune responses; immunomodulatory antibodies, including but not limited to, anti-CTLA4, anti-CD40, anti-41BB, anti-OX40, anti-PD1, anti-PDL1 and chemokines such as CCL2, CXCL1 and CXCL10.

Examples of diagnostic agents include fluorescent proteins or enzymes producing a colorimetric reaction. Exemplary proteins that generate a detectable signal include, but are not limited to green fluorescent protein (Genbank Accession No. AAL33912), alkaline phosphatase (Genbank Accession No. AAK73766), peroxidase (Genbank Accession No. NP_568674), histidine tag (Genbank Accession No. AAK09208), Myc tag (Genbank Accession No. AF329457), biotin ligase tag (Genbank Accession No. NP_561589), orange fluorescent protein (Genbank Accession No. AAL33917), beta galactosidase (Genbank Accession No. NM_125776), Fluorescein isothiocyanate (Genbank Accession No. AAF22695) and strepavidin (Genbank Accession No. S11540).

In another example, the diagnostic agent is a luminescent protein such as products of bacterial luciferase genes, e.g., the luciferase genes encoded by Vibrio harveyi, Vibrio fischeri, and Xenorhabdus luminescens, the firefly luciferase gene FFlux, and the like.

It will be appreciated that the present invention also contemplates expression of heterologous proteins that elevate sensitivity of imaging or screening with computed tomography (CT).

Recombinant methods for inserting heterologous sequences into a phage genome are well-known in the art. The appropriate coding sequence is inserted in one or more of several locations in the phage genome. In one embodiment, the nucleic acid insert that is introduced into the phage genome is approximately no more than 10% of the phage genome length.

The payload coding sequence is inserted either after early, middle or late expressing phage genes and it can be expressed as part of a phage operon, relying on either an existing phage operon, promoter and terminator, or as a distinct operon. In the latter case, a relevant promoter and terminator from the phage is inserted as part of the newly formed operon.

For example, if strong expression of a payload is required, the payload coding sequence is added after the stop codon of the major capsid protein and expressed as part of the major capsid operon. Alternatively, it can be expressed by addition of a major capsid protein promoter and terminator as an individual newly formed operon which can be inserted anywhere in the phage genome that would not damage the functionality of the phage. If low expression of a payload is desired, the payload coding sequence can be added after the terminase gene (or other low expressing gene), which usually has low expression. Moreover, payload levels are tuned by adding a ribosome binding site with a desired strength.

In one embodiment, the payload is expressed under control of a bacterial host (e.g. FN) native promoter, using the phage native promoter or a promoter belonging to another phage that is known to infect the host.

In order to avoid negatively affecting phage infectivity and specificity, the payload coding sequence is typically not inserted inside an existing phage open reading frame. An exception to this is the case when the payload is intended to be expressed as a fusion protein of the phage outer coat. In that latter case of payload display, the payload coding sequence is added in frame to sequence encoding the phage coat protein.

In still another embodiment, the heterologous sequence renders a temperate phage lytic (e.g. by disrupting a gene crucial for the lysogenic cycle such as the repressor gene).

The term “lytic bacteriophage” refers to a bacteriophage that infects a bacterial host and causes that host to lyse without incorporating the phage nucleic acids into the host genome. A lytic bacteriophage is typically not capable of reproducing using the lysogenic cycle.

The term “temperate bacteriophage” refers to a bacteriophage that is capable of reproducing using both the lysogenic cycle and the lytic cycle. Lysogeny is characterized by integration of the bacteriophage nucleic acid into the host bacterium's genome or formation of a circular replicon in the bacterial cytoplasm. In this condition the bacterium continues to live and reproduce normally.

In order to convert a temperate bacteriophage to a lytic bacteriophage (i.e. one not capable of using the lysogenic pathway), the repressor gene may be modified such that it is no longer functional. In one embodiment, the repressor gene (or part thereof) is deleted. Methods of modifying bacteriophage genes are known in the art and include cloning and homologous recombination techniques known in the art of molecular biology. Additional information is provided in “In-vitro translation transcription for phage “rebooting” (Mark Rustad, Allen Eastlund, Paul Jardine, Vincent Noireaux, Cell-free TXTL synthesis of infectious bacteriophage T4 in a single test tube reaction. Synthetic Biology, Volume 3, Issue 1, 2018, ysy002, doidotorg/10.1093/synbio/ysy002).

It will be appreciated that as well as genetically modifying the bacteriophage to express a payload, the present inventors also contemplate chemically attaching a payload to the phage outer coat. Such payloads may be protein based or non-protein based.

Exemplary methods of attaching a payload to the phage are described in Aggarwal, N., Hwang, I. Y. & Chang, M. W. Phage-boosted chemotherapy. Nat Biomed Eng 3, 680-681 (2019); Dong X., Pan P., Zheng D.-W., Bao P., Zeng X., Zhang X.-Z. Bioinorganic hybrid bacteriophage for modulation of intestinal microbiota to remodel tumor-immune microenvironment against colorectal cancer. Sci. dv. 2020; 6: eaba1590.; He X., Yang Y., Guo Y., Lu S., Du Y., Li J.-J., Zhang X., Leung N. L. C., Zhao Z., Niu G., et al. Phage-Guided Targeting, Discriminative Imaging, and Synergistic Killing of Bacteria by AIE Bioconjugates. J. Am. Chem. Soc; Dong S., Shi H., Zhang X., Chen X., Cao N., Mao C., Gao X., Wang L. Difunctional bacteriophage conjugated with photosensitizers for Candida albicans-targeting photodynamic inactivation. Int. J. Nanomed. 2018; 13:2199-2216; Anany H., Chen W., Pelton R., Griffiths M. W. Biocontrol of Listeria monocytogenes and Escherichia coli O157:H7 in Meat by Using Phages Immobilized on Modified Cellulose Membranes. Appl. Environ. Microbiol. 2011; 77:6379-6387; and Liana A. E., Marquis C. P., Gunawan C., Gooding J. J., Amal R. Antimicrobial activity of T4 bacteriophage conjugated indium tin oxide surfaces. J. Colloid Interface Sci. 2018; 514:227-233, the contents of which are incorporated herein by reference.

The bacteriophages described herein may be used to treat subjects having diseases associated with Fusobacterium nucleatum infection.

Thus, according to another aspect of the present invention, there is provided a method of treating a disease associated with a Fusobacterium nucleatum infection in a subject in need thereof, comprising administering to the subject a therapeutically effective amount of at least one isolated bacteriophage capable of infecting at least one Fusobacterium nucleatum bacterial species causing the infection, wherein the at least one bacteriophage has a genomic nucleic acid sequence at least 85% identical to the nucleic acid sequence as set forth in SEQ ID NOs: 1, 2, 3, 4, 5, 6, 7 or 8, thereby treating the disease associated with the Fusobacterium nucleatum infection.

According to another aspect of the present invention there is provided a method of treating a disease associated with a Fusobacterium nucleatum infection in a subject in need thereof, comprising administering to the subject a therapeutically effective amount of at least one isolated bacteriophage capable of infecting a Fusobacterium nucleatum bacterial species, wherein the bacteriophage has a genomic nucleic acid sequence at least 85% identical to the genomic nucleic acid sequence of a bacteriophage deposited with the DSMZ under deposit number DSM 33306, DSM 33307, DSM 33582, DSM 33583, DSM 33639, DSM 33640, DSM 33641, DSM 33642 or DSM 33644, thereby treating the disease associated with the Fusobacterium nucleatum infection.

As used herein, the term “subject” includes mammals, preferably human beings at any age which suffer from the pathology. Preferably, this term encompasses individuals who are at risk to develop the pathology.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the phrase “disease associated with a Fusobacterium nucleatum infection” refers to a disease which is associated with proliferation of Fusobacterium nucleatum bacteria.

Diseases associated with Fusobacterium nucleatum infection include diseases in which Fusobacterium nucleatum is present at a disease site (e.g. in a tumor). In one embodiment, the amount of Fusobacterium nucleatum is typically greater than the amount in a healthy subject.

Such diseases include cancer, periodontal diseases and inflammatory bowel disease.

Inflammatory bowel diseases (IBD) are severe gastrointestinal disorders characterized by intestinal inflammation and tissue remodeling, that increase in frequency and may prove disabling for patients. The major forms of IBD, ulcerative colitis (UC) and Crohn's disease are chronic, relapsing conditions that are clinically characterized by abdominal pain, diarrhea, rectal bleeding, and fever.

Exemplary cancers that can be treated with the bacteriophages described herein include, but are not limited to oral and esophageal cancer, colorectal cancer, pancreatic cancer, gastric cancer, gall bladder cancer, breast cancer, lung cancer, bladder cancer and melanoma.

According to a particular embodiment, the cancer is colorectal cancer.

The present invention contemplates treatment using more than one of the above disclosed phages, wherein at least two of the phage target a different Fusobacteria nucleatum subspecies.

In one embodiment, the treating comprises administering at least two, three or four of the above disclosed phages wherein each of the phages in the cocktail target a different Fusobacteria nucleatum subspecies.

In another embodiment, the treating comprises administering at least two different phages which target a different strain of Fusobacteria nucleatum.

In another embodiment, the treating comprises administering at least two, three, four, five or more of the above disclosed phages wherein each of the phages target a different strain of Fusobacteria nucleatum.

The bacteriophage may be used per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the bacteriophage accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include topical, oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient. In one embodiment, the bacteriophage may be administered directly into the tumor of the subject.

Pharmaceutical compositions of some embodiments of the invention may be manufactured by processes well known in the art, e.g., by means of conventional mixing, dissolving, granulating, dragee-making, levigating, emulsifying, encapsulating, entrapping, spray drying, coating or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (bacteriophage) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

In some embodiments, the pharmaceutical composition is delivered to a subject in need thereof so as to provide one or more bacteriophage in an amount corresponding to a multiplicity of infection (MOI) of about 1 to about 10. MOI is determined by assessing the approximate bacterial load in the tumor, or using an estimate for a given type of disease (cancer); and then providing phage in an amount calculated to give the desired MOI.

In some embodiments, MOI may be selected based on the “multiplicity of 10 rule”, which states that where there are on average in order of 10 phages adsorbed per bacterium, bacterial density reduces significantly (Abedon S T, 2009, Foodborne Pathog Dis 6:807-815; and Kasman L M, et al., 2002, J Virol 76:5557-5564); whereas lower-titer phage administration (e.g., using a MOI lower than 10) is unlikely to be successful (Goode D, et al., 2003, App Environ Microbiol 69:5032-5036; Kumari S, et al., 2010, J Infect Dev Ctries 4:367-377).

In other embodiments, the amount of phage is provided so as to reduce the amount of bacteria (e.g. Fusobacterium nucleatum) present in the tumor by at least 10%, 20%, 30%, 40%, 50%, 60%, 70%, 80%, 90% or even 100%.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Compositions described herein may comprise more than one phage strain. In one embodiment, the composition comprises 2 phage strains, 3 phage strains, 4 phage strains, 5 phage strains or more.

In one embodiment, the bacteriophage cocktails comprise phages that target a single Fusobacteria nucleatum subspecies i.e. only Fusobacteria nucleatum nucleatum subspecies, only Fusobacteria nucleatum animalis subspecies, only Fusobacteria nucleatum vincentii subspecies, or only Fusobacteria nucleatum polymorphum subspecies.

In one embodiment, the bacteriophage cocktails comprise phages that target more than one Fusobacteria nucleatum subspecies.

Thus, for example a bacteriophage cocktail may comprise at least two phages which target a different Fusobacteria nucleatum subspecies.

Alternatively, the bacteriophage cocktail may comprise at least two, three or four phages wherein each of the phages in the cocktail target a different Fusobacteria nucleatum subspecies.

In another embodiment, the bacteriophage cocktails comprise at least two different phages targeting a different strain of Fusobacteria nucleatum.

Alternatively, the bacteriophage cocktail may comprise at least two, three, four, five or more phages wherein each of the phages in the cocktail target a different strain of Fusobacteria nucleatum.

The pharmaceutical compositions of the present invention also may be combined with one or more non-phage therapeutic and/or prophylactic agents, useful for the treatment and/or prevention of bacterial infections, as described herein and/or known in the art (e.g. one or more traditional antibiotic agents). Other therapeutic and/or prophylactic agents that may be used in combination with the phage(s) or phage product(s) of the invention include, but are not limited to, antibiotic agents, anti-inflammatory agents, antiviral agents, antifungal agents, or local anesthetic agents. In some preferred embodiments, the pharmaceutical composition is formulated for treatment of cancer (e.g. intratumoral formulation) and comprises one or more additional therapeutic and/or prophylactic agents selected from anti-cancer agents antibiotic agents, antifungal agents, and local anesthetic agents. In some embodiments, the pharmaceutical composition comprises a phage cocktail combination of the invention, which is administered in the absence of a standard or traditional antibiotic agent.

Anti-cancer drugs that can be co-administered with the bacteriophages of the invention include, but are not limited to Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Standard or traditional antibiotic agents that can be administered with the bacteriophages described herein include, but are not limited to, amikacin, gentamicin, kanamycin, neomycin, netilmicin, paromomycin, rhodostreptomycin, streptomycin, tobramycin, apramycin, rifamycin, naphthomycin, mupirocin, geldanamycin, ansamitocin, carbacephems, imipenem, meropenem, ertapenem, faropenem, doripenem, panipenem/betamipron, biapenem, PZ-601, cephalosporins, cefacetrile, cefadroxil, cefalexin, cefaloglycin, cefalonium, cefaloridine, cefalotin, cefapirin, cefatrizine, cefazaflur, cefazedone, cefazolin, cefradine, cefroxadine, ceftezole, cefaclor, cefonicid, cefprozil, cefuroxime, cefuzonam, cefmetazole, cefotetan, cefoxitin, cefcapene, cefdaloxime, cefdinir, cefditoren, cefetamet, cefixime, cefmenoxime, cefteram, ceftibuten, ceftiofur, ceftiolene, ceftizoxime, ceftriaxone, cefoperazone, ceftazidime latamoxef, cefclidine, cefepime, cefluprenam, cefoselis, cefozopran, cefpirome, cefquinome, flomoxef. ceftobiprole, azithromycin, clarithromycin, dirithromycin, erythromycin, roxithromycin, aztreonam, pencillin and penicillin derivatives, actinomycin, bacitracin, colistin, polymyxin B, cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, garenoxacin, gemifloxacin, stifloxacin, trovalfloxacin, prulifloxacin, acetazolamide, benzolamide, bumetanide, celecoxib, chlorthalidone, clopamide, dichlorphenamide, dorzolamide, ethoxyzolamide, furosemide, hydrochlorothiazide, indapamide, mafendide, mefruside, metolazone, probenecid, sulfacetamide, sulfadimethoxine, sulfadoxine, sulfanilamides, sulfamethoxazole, sulfasalazine, sultiame, sumatriptan, xipamide, tetracycline, chlortetracycline, oxytetracycline, doxycycline, lymecycline, meclocycline, methacycline, minocycline, rolitetracycline, methicillin, nafcillin, oxacilin, cloxacillin, vancomycin, teicoplanin, clindamycin, co-trimoxazole, flucloxacillin, dicloxacillin, ampicillin, amoxicillin and any combination thereof.

Standard antifungal agents include amphotericin B such as liposomal amphotericin B and non-liposomal amphotericin B.

The bacteriophage and bacteriophage cocktails of the invention can be used in anti-infective compositions for controlling the growth of bacteria, in particular Fusobacterium nucleatum, in order to prevent or reduce the incidence of nosocomial infections. The anti-infective compositions find use in reducing or inhibiting colonization or growth of bacterial on a surface contacted therewith. The bacteriophages of the invention may be incorporated into compositions that are formulated for application to biological surfaces, such as the skin and mucus membranes, as well as for application to non-biological surfaces.

Anti-infective formulations for use on biological surfaces include, but are not limited to, gels, creams, ointments, sprays, and the like. In particular embodiments, the anti-infective formulation is used to sterilize a surgical field, or the hands and/or exposed skin of healthcare workers and/or patients.

Anti-infective formulations for use on non-biological surfaces include sprays, solutions, suspensions, wipes impregnated with a solution or suspension and the like. In particular embodiments, the anti-infective formulation is used on solid surfaces in hospitals, nursing homes, ambulances, etc., including, e.g., appliances, countertops, and medical devices, hospital equipment. In preferred embodiments, the non-biological surface is a surface of a hospital apparatus or piece of hospital equipment. In particularly preferred embodiments, the non-biological surface is a surgical apparatus or piece of surgical equipment.

The present invention also encompasses diagnostic methods for determining the causative agent at the site of the bacterial infection. In certain embodiments, the diagnosis of the causative agent of a bacterial infection is performed by (i) culturing a sample from a patient, e.g., a tumor biopsy or other sample appropriate for culturing the bacteria causing the infection; (ii) contacting the culture with one or more bacteriophages of the invention; and (iii) monitoring for evidence of cell growth and/or lysis of the culture. Because the activity of phages tends to be species or strain specific, susceptibility, or lack of susceptibility, to one or more phages of the invention can indicate the species or strain of bacteria causing the infection.

The sample may be a tissue biopsy or swab collected from the patient, or a fluid sample, such as blood, tears, or urine.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion. General references are provided throughout this document. The procedures therein are believed to be well known in the art and are provided for the convenience of the reader. All the information contained therein is incorporated herein by reference.

### Example 1

### Isolating and Characterizing Phage

Using the methodology described in Machuca et al., [App Env Microbiol, 2010 November; 76(21):7243-50] on fresh saliva samples, 10 new phage have been isolated, including phage against each of the 4 different FN subspecies—vincentii, animalis, nucleatum and polymorphum. The host bacteria used to isolate phage, the subspecies against which they demonstrate infectivity and phage characteristics are shown below in Table 1. All assessments of infectivity were carried by applying 5 μl of phage at 10⁶ pfu/mL of phage on a bacterial lawn of the bacteria being used for screening. The bacterial lawn was inspected for appearance of clear plaques which indicate amplification of phage on the bacteria. The phages are each further described below.

TABLE 1
Exemplary Isolated Bacteriophage against FN Subspecies.

| | | | | | Tissue | | | | |
|---|---|---|---|---|---|---|---|---|---|
| | DNA/ | Temperature/ | | | source of | Also | Additional | SEQ ID | Deposit |
| Name | RNA | Lytic | Infects | Host | host | recognizes | information | NO: | no. |
| FN1-1 | DNA | T | FN1 | CCUG 32880/ | sinusitis | | | 1 | DSM |
| | | | (vincentii) | ATCC 51190 | of upper | | | | 33306 |
| | | | | (DSMZ DSM | jaw | | | | |
| | | | | 33250) | | | | | |
| FN14-1 | DNA | T | FN14 | Genbank accession | colonic | | | 2 | DSM |
| | | | (vincentii) | GCA_000347315.1 | tumor | | | | 33639 |
| FN14-2 | DNA | L | FN14 | Genbank accession | colonic | FN2 | | 3 | DSM |
| | | | (vincentii) | GCA_000347315.1 | tumor | (vincentii) | | | 33640 |
| FN14-3 | DNA | L | FN14 | Genbank accession | colonic | | | 4 | DSM |
| | | | (vincentii) | GCA_000347315.1 | tumor | | | | 33641 |
| FN14-4 | DNA | L | FN14 | Genbank accession | colonic | | | 5 | DSM |
| | | | (vincentii) | GCA_000347315.1 | tumor | | | | 33642 |
| FN14-6 | DNA | T | FN14 | Genbank accession | colonic | | | 6 | DSM |
| | | | (vincentii) | GCA_000347315.1 | tumor | | | | 33644 |
| FN2-58* | RNA | L | FN2 | CCUG 37843/ | inflamed | FN8 | 3 | 15 | DSM |
| | | | (vincentii) | ATCC 49256 | gingiva | (nucleatum) | genomic | 16 | 33582 |
| | | | | | | | segments | 17 | |
| FN5-1 | DNA | T | FN5 | BEI HM-992 | colonic | | | 7 | DSM |
| | | | (animalis) | (DSMZ DSM | tumor | | | | 33307 |
| | | | | 33251) | | | | | |
| FN7-1 | RNA | L | FN7 | BEI HM-74 | normal | FN8 | 3 | 18 | DSM |
| | | | (animalis) | | colon | (nucleatum); | genomic | 19 | 33583 |
| | | | | | | FNC1*** | segments | 20 | |
| | | | | | | (polymorphum); | | | |
| | | | | | | FNC2*** | | | |
| | | | | | | (polymorphum) | | | |

*Same saliva host;
** Derived from FN7-1;
***FNC strains from BiomX saliva.

As can be seen from Table 1, each of the four F. nucleatum subspecies is targeted by multiple phages of the disclosure. FN subspecies vincentii is targeted by the first 7 phages listed (including 2 from the same donor). FN subspecies animalis is targeted by FN5-1 and FN7-1. FN subspecies nucleatum is targeted by FN7-1, FN2-58. FN subspecies polymorphum is targeted by FN7-1.

Also, some of phages are temperate, as determined by sequence analyses that revealed the presence of integrases required for lysogenic integration. Three of the phages are double stranded (DS) lytic RNA phages. Lytic RNA phages against FN have never previously been reported. Two of the RNA phages have a broad target host range under the present conditions for examining sensitivity to phage infectivity and can infect representatives of more than a single FN subspecies.

DNA Phages

1. Phage FN1-1—Double Stranded DNA Temperate Phage:

Phage FN1-1 (deposit number DSM 33306), isolated on FN1 Fusobacterium nucleatum subsp. vincentii (CCUG 32880/ATCC 51190, DSMZ DSM 33250), is a double stranded temperate DNA phage with a length of about 36,000 bp. The phage is packed by a cos packaging mechanism with the sequence SEQ ID NO: ATACCGCTTCCCCTCTTT (SEQ ID NO: 8) based on Oxford Nano-pore sequencing and PhageTerm [JR Garneau, et al., Sci Rep 2017 Aug. 15, 7(1):8292]analysis).

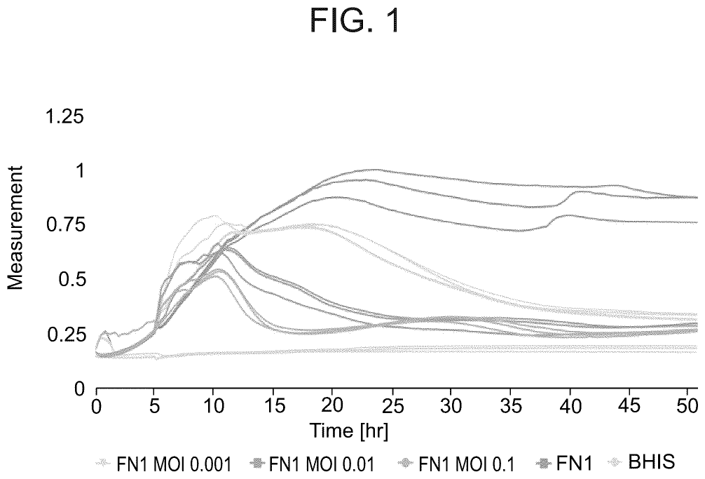

FN1-1 has 52 open reading frames (ORFs) above 80 amino acids in length out of which only 8 are identified by BLASTX. One of these 8 genes is an integrase gene which indicates that this phage is a temperate phage. While temperate, FN1-1 causes a collapse of the host growth curve as shown in FIG. 1.

FN1-1 infection kinetics at different Multiplicities of Infection (ratios of phage to target bacteria) in liquid culture are shown in FIG. 1. A control sample with only BHIS buffer results in OD reading close to zero.

2. Phage FN14-1—Double Stranded DNA Temperate Phage

Phage FN14-1 (deposit number DSM 33639) is a double stranded DNA temperate phage with a genome length of about 39,000 bp that was isolated on FN14 Fusobacterium nucleatum subsp. vincentii GenBank accession GCA_000347315.1.

The phage encodes 59 ORF above 80 amino acids in length. An integrase gene and a repressor gene were identified.

3. Phage FN14-2—Double Stranded DNA Lytic Phage

Phage FN14-2 (deposit number DSM 33640) is a double strand DNA lytic phage with a genome length of about 126,000 bp. The phage was isolated on FN14 Fusobacterium nucleatum subsp. vincentii Genbank accession GCA_000347315.1 and is able to infect both its original host and FN2 F. nucleatum subsp. vincentiii (CCUG 37843/ATCC 49256).

4. Phage FN14-3—Double Stranded DNA Lytic Phage

Phage FN14-3 (deposit number DSM 33641) is a double strand DNA lytic phage with a genome length of about 80,000 bp that was isolated on FN-14 Fusobacterium nucleatum subsp. vincentii Genbank accession GCA_000347315.1. No integrase gene was detected in the sequence of FN14-3.

5. Phage FN14-4—Double Strand DNA Lytic Phage

Phage FN14-4 (deposit number DSM 33642) is a double stranded DNA lytic phage with a genome length of about 125,000 bp that was isolated on FN14-4 Fusobacterium nucleatum subsp. vincentii Genbank accession GCA_000347315.1. No integrase gene was detected in its genome.

6. Phage FN14-6—Double Stranded DNA Temperate Phage

Phage FN14-6 (deposit number DSM 33644) is a double stranded DNA temperate phage with a genome length of about 38,000 bp was isolated on FN14 Fusobacterium nucleatum subsp. vincentii Genbank accession GCA_000347315.1. An integrase gene and a repressor gene was detected

7. Phage FN5-1—Double Stranded DNA Temperate Phage

Phage FN5-1 (deposit number DSM 33307) is a double stranded DNA phage with a genome length of about 39,000 bp that was isolated on FN5 Fusobacterium nucleatum subsp. animalis (BEI HM-992, DSMZ DSM 33251). Its genome has 58 ORF above 80 amino acids in length. An integrase gene was detected and integration into the genome was confirmed. The phage integrates into the host genome between the host copper amine oxidase (GenBank: PGH26162.1) gene and endonuclease MutS2 [Fusobacterium nucleatum] (GenBank: PIM90054.1) gene. A repressor, also required for lysogeny, was identified.

B. Double-Stranded RNA Phages:

Each of the three RNA double stranded phages (FN2-58 and FN7-1) contains 3 chromosomes of about 3.5 KB, 4.5 KB and 7 KB in length. This is the first known disclosure of an RNA phage against Fusobacterium subsp. nucleatum. BLAST analysis of the phages' contigs did not yield any specific hits but a few random hits of about 50 bp homology regions to bacterial genomes.

1. Phage FN7-1 Double-Stranded RNA Lytic Phage

Phage FN7-1 DS RNA (DSM 33583) was isolated on FN7 F. nucleatum subsp. animalis (BEI HM-74) and also infects FN8 F. nucleatum subsp. nucleatum (BEI HM-993) and FNC1 (F. nucleatum subsp. polymorphum, DSMZ DSM 33248). Phage FN7-1 also infects FNC2 (F. nucleatum subsp. polymorphum, DSMZ DSM 33249) at a lower efficiency (Efficiency of Plating (“EOP”) of 0.01 compared to EOP of 1 on FN7 (i.e., two orders of magnitude lower than on FN7).

2. Phage FN2-58 Double-Stranded RNA Lytic Phage

FN2-58 DS RNA phage (Deposit No. DSM 33582) was isolated on FN2 F. nucleatum subsp. vincentii (CCUG 37843/ATCC 49256). FN2-58 infects FN2 at an EOP of 0.01 and infects FN8 at an EOP of 0.01. FN2-58 has 95% identity to FN7-1 phage.

### Example 2

Demonstration of Phage Targeting to FN Bacteria-Bearing Tumors

The phages of the present disclosure were shown to target intra-tumorally located FN bacteria. A syngeneic subcutaneous mouse tumor model was utilized to assess the efficacy of phage delivery into F. nucleatum bearing tumors.

Syngeneic Animal Tumor Model

A syngeneic subcutaneous model was utilized in this study. One week prior to study initiation, murine colon carcinoma CT26 cells (ATCC CRL-2638) were thawed and cultured. On day 0, cells were trypsinized, centrifuged and resuspended in phosphate buffered saline (PBS). A dose of 5×10⁵ cells/mouse in 0.1 mL was injected subcutaneously to the right flank of female Balb/c mice. Animals were observed for tumor appearance and tumors were measured for parameters of length (L) and width (W) using a caliper, three times a week until study termination.

Preparation and administration of F. nucleatum bacteria (FN5): FN5 (BEI HM-992) was cultured from a frozen stock in BHIS media supplemented with 0.1% L-cysteine on an anaerobic BHIS plate. Incubation was carried out in Lock & Lock Boxes at 37° C. inside an aerobic incubator for 2-3 days. A starter culture, prepared from the plate, was diluted 1:100 into fresh media, and incubated overnight. The next morning bacteria were prepared by centrifugation and resuspension in PBS.

Administration of bacteria into mice was carried out after group allocation, so that mice with the largest or the smallest tumors were excluded from the study, and the rest of the animals were inoculated with F. nucleatum FN5 via slow (0.2 mL/minute) tail vein intravenous (IV) injection. Bacteria were administered on day 12 after tumor induction. Different CFU doses (5×10⁶, 5×10⁷ and 5×10⁸) were tested to identify the optimal dose for tumor colonization.

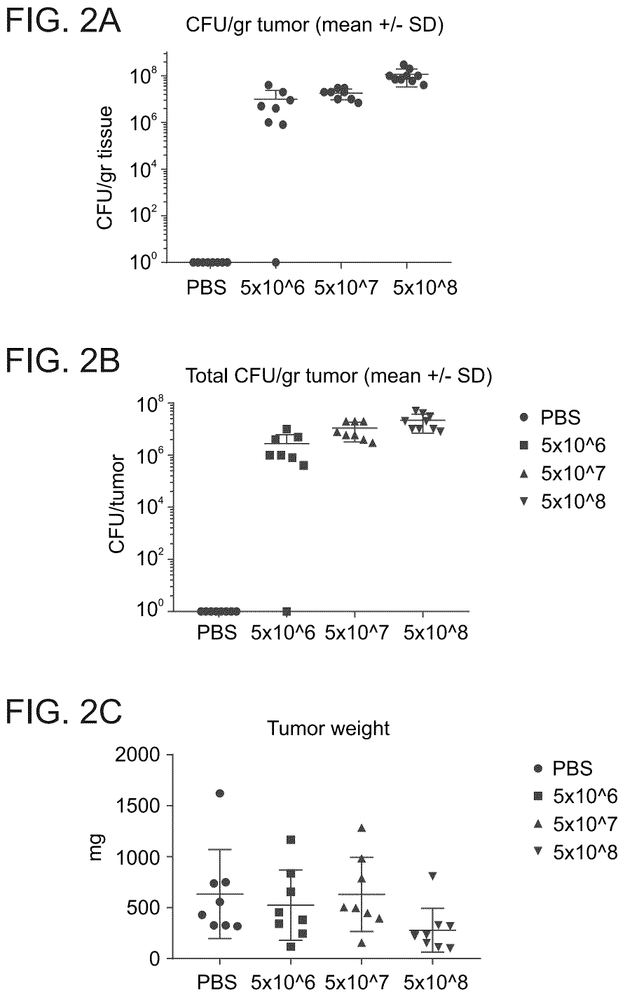

F. nucleatum FN5 was incorporated into subcutaneous murine colon carcinoma tumors (CT26) following IV injection and survival of bacteria in the tumor environment was shown. Slowly injected F. nucleatum bacteria showed co-localization in a dose dependent manner (FIGS. 2A and 2B). Injection of the highest dose, 5×10⁸ CFU/animal, inhibited growth of the CT26 derived tumor (FIG. 2C). An intermediate dose of 5×10⁷ CFU/animal was selected for further studies.

Preparation and administration of FN5-1 phage: For administration of phage, 300 ml of a filtered lysate was concentrated using a 100 KDa Centrifugal Filter Unit (Amicon UFC910024) to a final volume of 2 ml. Removal of endotoxins was carried out using Hyglos EndoTrap HD Resin with the manufacturer's protocol. Buffer exchange was carried out to transfer purified phage at a concentration of 7.5×10¹¹ PFU/mL into SM-buffer (100 mM NaCl, 50 mM MgSO₄, 25 mM Tris-HCl, LAL water) at a final phage concentration of 6.5×10⁹. Endotoxin test showed that phage was endotoxin free (<0.05 EU/mL, using Endosafe®-PTS™ instrument).

FN5-1 phage (in 50 mM Tris-HCl, 100 mM NaCl, 5 mM MgCl₂, 0.1 mM MnCl₂ pH=7.5 and provided at a titer of 10⁹ PFU/mL) were administered by IV injection one day following FN5 dosing to the CT26 tumor-containing mice via slow tail vein injection (0.2 mUanimal) (FIG. 3A). Selected organs were collected, weighed, and processed for colony forming units (CFU) count, or for qPCR as described below.

Tumors were harvested 24 hours following phage administration, weighed, and processed for colony forming units (CFU) count, or for qPCR as described below. Phage were shown to home to tumors and integrate into the FN bacteria incorporated therein. Detection of lysogenized intra-tumor F. nucleatum by qPCR using one set of primers in the phage SEQ ID NO: 9 CTATTTGTTGGCTACCTAATTAAG and one in the bacterial DNA adjacent to the integration site SEQ ID NO: 10 AAATACTATCCTGACTGGTGTAAG (FIG. 3B) were used to detect integration of FN5-1 into the FN5 bacteria. The probe sequence utilized was SEQ ID NO: 11 TAGCCATCTGGATCTGTTAGAATT.

The amount of temperate phage integrated into the FN genome was determined for both FN5-1 phage treated and control animals after normalizing to the total FN content. FN content was determined by detection of the NusG gene (GenBank accession AAL94126.1), a unique gene of F. nucleatum with no match to any gene of any other species, by qPCR. qPCR of NusG gene was carried out by utilizing the following forward primer, reverse primer & probe: SEQ ID NO:12 CCTCGTGTATGGTATGAAGT, SEQ ID NO:13 ATCAACCATTACTTTAACTCTAC, and SEQ ID NO:14 ATCAAGAAGGACAAGTTGCTGA, respectively. Statistical analysis was performed using a two-tailed t-test. A statistically significant increase (P<0.0001) in temperate phage was observed in tumors derived from mice to which both FN and phage had been administered compared to mice that received FN only (FIG. 3C).

### Example 3

### Conversion of a Temperate Phage to a Lytic Phage

The conversion of a temperate phage of this disclosure into a lytic phage may be achieved by a complete deletion of the repressor present in the temperate phage. This technique yields lytic phages that are useful for lysing and killing various FN subspecies that are associated with a disease or condition.

One way to achieve the conversion of a temperate phage to a lytic phage is by engineering the phage genome in Saccharomyces cerevisiae: a temperate E. coli lambda phage was converted into an obligatorily lytic phage by complete deletion of the repressor and re-assembly of its genome in yeast. The lambda phage genome was divided into 8 parts of approximately 6 KB each by PCR. The CI repressor gene was not amplified. The phage was assembled inside yeast on the backbone of a pYES1L vector (GenArt) as demonstrated by the protocol described in H. Ando et al., Cell Sys 2015 Sep. 23; 1(3):187-96. The assembled plasmid was then obtained by miniprep from yeast and transformed into electrocompetent E. coli 10G cells (Lucigen) to produce viable phage as described in H. Ando et al. The new phage with the repressor deletion exhibited the same infection kinetics as the Lambda Vir mutant, a known lytic mutant with a mutation in its operator sequences (FIG. 4).

Another way to achieve the conversion of a temperate phage to a lytic phage is by disrupting at least one gene essential for the lysogenic cycle, as described in Example 4.

### Example 4

### Insertion of a Payload into a Phage and Disruption of Integrase

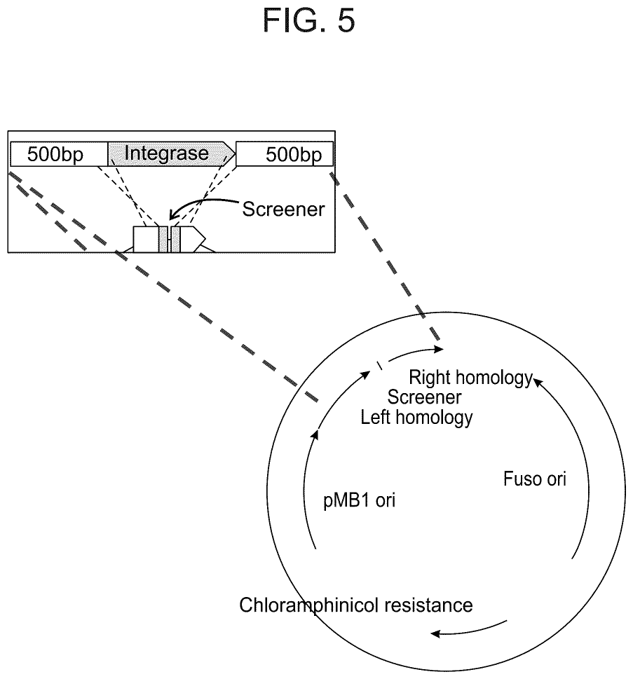

A screenable 8 bp foreign DNA payload segment that can be detected by PCR (a “screener”) was inserted into an FN temperate phage inside its integrase gene in such a way as to inactivate this gene and make the phage obligatorily lytic. Homologous recombination was used. An FN shuttle plasmid was generated as shown in FIG. 5 by combining a chloramphenicol resistance gene, FN origin of replication from a native FN plasmid and E. coli origin of replication (pMB1). The 500 bp flanking the integrase gene on either side was also cloned into the construct to promote homologous recombination. An FN strain was transformed with this plasmid and infected with an FN temperate phage at MOI 0.1 to promote homologous recombination. The lysate of this infection was used to infect an additional FN strain that does not contain a plasmid. The lysate that was produced was screened by PCR to validate the presence of the recombinant payload bearing phage. Following such validation, individual plaques were tested for the presence of the “screener” to identify the recombinant lytic payload bearing phage.

### Example 5

### Identification of FN Subspecies

Identification of which FN subspecies is present in a tumor of each patient was carried out by PCR of the NusG gene followed by sequencing of the amplicon. Alignment of the sequence of the approximately 112 bp NusG amplicon in representatives of each subspecies (10 nucleatum, 22 animalis, 21 polymorphum and 13 vincentii bacteria) revealed single nucleotide differences that can be used to distinguish between them.

In addition, targeted next generation sequencing (NGS) of the NusG gene was applied to quantify the relative abundance of F. nucleatum subspecies in tumor and normal tissues. Combined with qPCR data, the absolute abundance for each of the subspecies was determined. With this data, the present inventors show that F. nucleatum subspecies animalis is the most prevalent subspecies in colorectal cancer tumors. Moreover, it was determine that it is a good candidate to be targeted by F. nucleatum phages given that at each absolute abundance cutoff it is consistently present in more tumors than the other subspecies.

### Example 6

Fusobacterium can Express Anti-Cancer Heterologous Gene

The amino acid sequences of the murine (Asn49Ser162, NCBI accession #P48346) and human (Asn49Ser162, NCBI accession #P40933) Interleukin-15 (IL-15) and cytosine deaminase were optimized for expression in Fusobacterium (using codon optimization bioinformatic tools) and cloned into the Fusobacterium expression vector MBQ. Cytosine deaminase converts the nontoxic prodrug 5-fluorocytosine (5-FC), which is used in cancer therapy, into its active and lethal. 5-fluorouracil (5-FU).

MBQ plasmid was generated by combining a native promoter of FN-14, Thiamphenicol-resistance gene, FN origin of replication from a native FN plasmid and E. coli origin of replication (pMB1). The murine and human Il-15 expression vectors were electroporated into competent Fusobacterium 14 (FN-14). Thiamphenicol-resistant colonies were selected, and plasmid integrity and purity were validated by next generation sequencing (NGS).

In order to verify that the full protein is expressed, the recombinant FN-14 cells were grown over night in 4 ml Brain heart infusion-supplemented (BHIS) medium (supplemented with 30 μg/ml Thiamphenicol), at 37° C. under anaerobic conditions. Bacteria were lysed by resuspension with 5% SDS and 100 mM Tris, and incubated at 100° C. for 5 min. The lysate samples were digested by trypsin and analyzed by LC-MS/MS using the Thermo Scientific™ Q Exactive™ Mass Spectrometer. Samples were identified by Thermo Scientific™ Proteome Discoverer Software using human/murine Il-15 sequences and bacterial FN14 databases. The MS analysis showed that the expected IL15 protein and the cytosine deaminase was fully expressed in FN-14 cells.

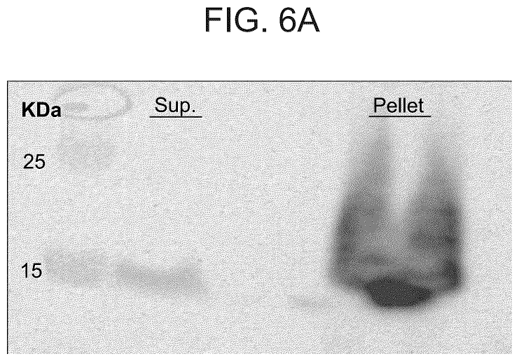

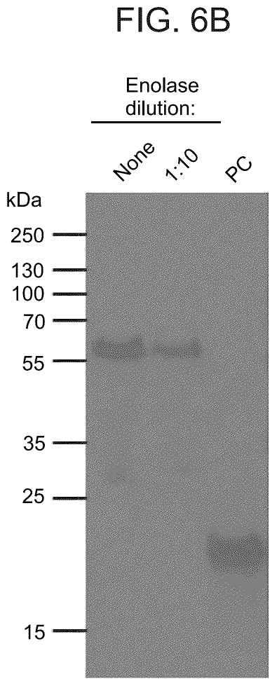

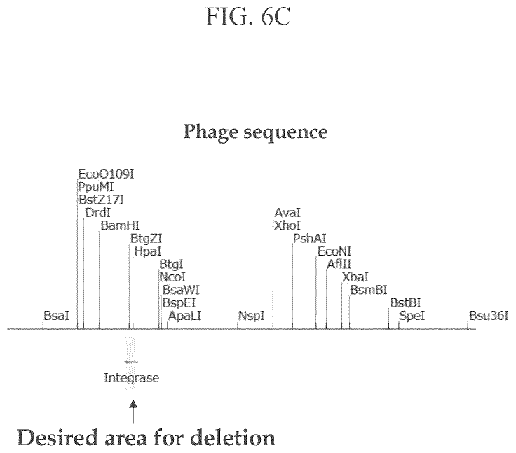

Fusobacterium cells were then transformed with an FN plasmid containing mouse IL-15 gene conjugated to C-terminal HiBiT tag and antibiotic resistance gene. Resistance colonies were selected and the presence of the plasmid was verified by NGS. The plasmid containing-FN cells were lysed using BugBuster Protein Extraction Reagent (Novagen), and IL-15-HiBiT expression was validated by both Western blot analysis (FIG. 6A) and luminescent assay using the Nano-Glo® Luciferase Assay (Promega) (Table 2A). For Western blot analysis, FN cell lysate (both supernatant and pellet fractions) was run on 4-12% gradient gels under denaturing conditions. After transfer to 0.45 μm nitrocellulose, the blotted bands were immunodetected with a Nano-Glo® HiBiT Blotting System (Promega).

TABLE 2A

| Sample | Luminescence signal (RLU) |
|---|---|
| F. nucleatum and IL15 | 112,117 |
| F. nucleatum control | 37 |

Enolase and pyruvate kinase are two native FN-14 proteins that are expressed at relatively high levels (based on MS analysis results). The genes encoding enolase and pyruvate kinase, together with their native promoters (˜500 bp downstream to the gene sequence) were cloned into Fuso plasmid. To facilitate detection, a C-terminal HiBiT tag was fused to the two proteins. The indicated expression vectors were electroporated into competent Fusobacterium 14 (FN-14) and Thiamphenicol-resistant colonies were selected. The expression of HiBiT-tagged pyruvate kinase and enolase was validated by Western blot (FIG. 6B and Table 2B, herein below).

TABLE 2B

| | Dilution | | | | |
|---|---|---|---|---|---|
| | 1:2 | 1:20 | 1:200 | 1:2,000 | 1:20,000 |
| NC | 126 | 120 | 120 | 120 | 120 |
| Enolase | 2,001,868 | 161,051 | 14,406 | 1,574 | 229 |
| Pyruvate kinase | 1,047,408 | 58,672 | 6,916 | 765 | 193 |

### Example 7

### Phage can be Engineered to Deliver a Heterologous Gene for Expression in a Fusobacterium Target

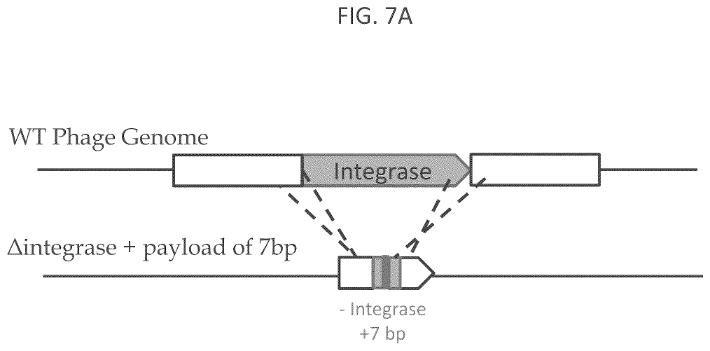

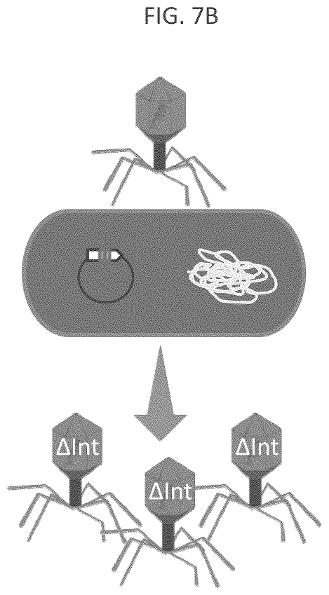

The Integrase gene of FN-14-1 phage was tagged at the C-terminal with three repeats of Flag tag or HiBiT tag using homologous recombination; To facilitate recombination, the last 500 bp of FN14-1 Integrase gene were cloned into the MBQ plasmid (see Example 6) upstream to the sequence encoding either the three repeats of Flag-tag or the HibiT tag, followed by a stop codon, and 500 bp stretch homolog to the sequence downstream of the FN-14-1 Integrase gene (FIGS. 6C and 7A). The recombination cassettes were electroporated into competent FN-14 bacterial cells. Thiamphenicol-resistant colonies were selected, and the plasmid integrity and purity were validated by NGS.

Homologous recombination events between the native FN-14-1 phage Integrase gene and the indicated recombination cassette were induced by infection of the plasmid-transformed FN-14 cells with FN-14-1 phage; The bacteria were grown in 4 ml BHIS medium (supplemented with 30 μg/ml Thiamphenicol) at 37° C. until OD of 0.2 at 600 nm was reached. The bacteria were infected with FN14-1 phage (MOI: 0.01) and incubated over night at 37° C. under anaerobic conditions. Thereafter, the bacteria were centrifuged (10000 g for 2 minutes), and the phage were harvested using a 0.2 μm filter.

Next, the phage samples, composed of a mixture of WT and recombinant FN-14-1 phages were used for infection of wild type FN-14 bacteria. Following infection, the expression of the Flag or Hibit tagged Integrase in wild-type FN-14 bacteria was detected using a luminescence assay, as summarized in Table 3A.

TABLE 3A

| Phage sample | Luminescent signal (RLU) |
|---|---|
| WT FN14-1 | 880 (Background) |
| Recombinant FN-14 (#1 Enrichment) | 15544 |
| Recombinant FN-14 (#2 Enrichment) | 22373 |

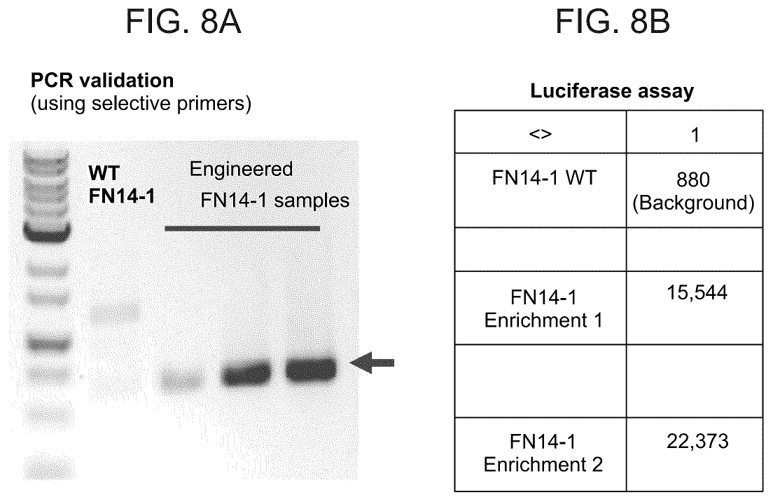

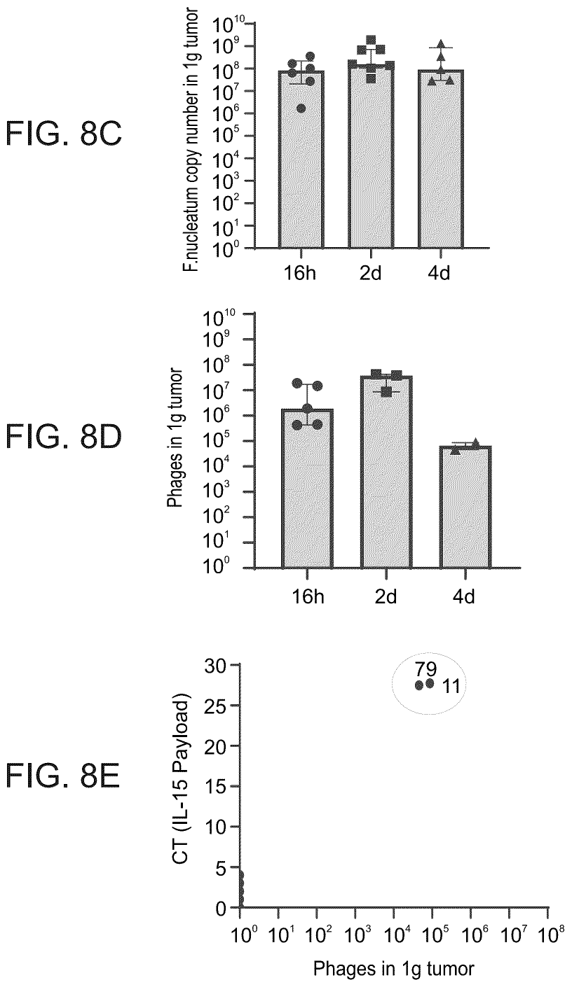

The recombinant phage (expressing an HiBiT-tagged integrase) was detected by PCR (using a selective primer pair for the recombinant phage), as illustrated in FIGS. 8A-B.

Synthetic biology approaches were next used to engineer Fusobacterium nucleatum targeting phage genetically modified to express a tagged IL-15 gene.

The mouse IL-15 gene, conjugated to C terminal HiBiT tag, was added to Fusobacterium nucleatum targeting phage using homologous recombination technique. Briefly, the IL-15-HiBiT gene was cloned into a pre-prepared plasmid containing appropriate regions up and downstream of the recombination site within the phage genome. Thereafter the plasmid containing FN cells were infected with the phage to facilitate homologous recombination. The engineered phage was isolated and then used to infect WT FN cells. IL-15 expression was validated in FN bacterial lysate by both Western blot analysis (FIG. 8C) and luminescent assay (Table 3B).

TABLE 3B

| Sample | Luminescence signal (RLU) |
|---|---|
| IL15 engineered phage lysate | 3,140,905 |
| WT phage lysate | 48 |

The following experiment was performed to show that phages engineered to express a heterologous protein (e.g. enolase tagged with HibiT) can successfully colonize a tumor in an animal model.

Materials and Methods:

A mouse model of colorectal cancer was established by subcutaneous injection of CT-26 cells to flank. The expression of Enolase-HiBit in tumors treated with an engineered phage was detected by a luminescent assay. Briefly, the proteins were purified from the tumors using a gentle MACS instrument by shredding the tumor in PBS-tween supplemented with protease inhibitors cocktail. A luminescent signal was measured in lysates prepared from tumors infected with either WT or engineered phage. Luminescence was detected using the Nano-Glo® Luciferase Assay (Promega).

Results

The results are summarized in Table 3C herein below.

TABLE 3C

| | Termination (time past | | |
|---|---|---|---|
| Phage admin. | phage administration) | | Pg payload/mg tumor |
| Native phage (control) | 16 | hours | 0 |
| Enolase phage | 16 | hours | 400 |
| Enolase phage | | | 370 |
| Enolase phage | | | 80 |
| Enolase phage | 4 | days | 27,810 |

| | |
|---|---|
| Enolase phage | 33,800 |

The following experiment was performed to show that phages engineered to express a heterologous protein (mouse IL-15) can successfully colonize a tumor in an animal model.

Materials and Methods:

A mouse model of colorectal cancer was established by subcutaneous injection of CT-26 cells to flank. Tumors were successfully colonized by FN following IV-administration. 4 days post bacteria inoculum, IL-15 engineered phage was IV-administrated and following 16 h, 2 days or 4 days the study was terminated.

Tumors were extracted, cut in cold PBS into 1-2 mm band, dissociated and subjected to bacteria and phage quantification. Lysates were centrifuged, and the supernatant containing the phages were filtered. The pellet containing the bacteria was used for gDNA extraction.

Samples were digested with proteinase K and incubated until full capsid digestion. Then, the gDNA was extracted as described by manufacture (D3012 Quick-DNA 96 kit). qPCR assay for detecting and quantity fusobacterium in gDNA samples was performed by detection of the Nusg gene. Quantification was performed using an established standard curve.

Double-layer plaque assay was used to quantify bacteriophage isolates. A starter culture of 4 mL BHIS was inoculated with 50-100 colonies from a plate. This culture was incubated at 37° C. for 16 hours in an anaerobic environment. A volume of 300 μL of this culture was mixed with 5 mL of BHIS top agar (pre-molten 0.4% agar BHIS supplemented with 1 mM Ca²⁺, Mn²⁺ and Mg²⁺ ions), and the mixture was poured over a BHIS bottom agar plate (1.5% agar BHIS). The plates were allowed to gel at room temperature. Then, tumor lysate was serially diluted using phage buffer, 5 μL of each dilution was dropped over the bacterial lawn. Then the plates were incubated for 16 hours at 37° C. in anaerobic environment until plaques are identified. For IL-15 detection, ˜30 mg of the tumor was processed. To avoid phage derived payload DNA, host DNA was removed by DNAse. Then, RNA was extracted, and cDNA was synthesed. Detection of IL15 was carried out using specific primers for engineered IL15 using qPCR.

Results

The results are illustrated in FIGS. 8C-E. FIG. 8C shows bacteria colonization in tumors. FIG. 8B shows the level of IL-15 engineered phage in tumors. FIG. 8C illustrates the results of qPCR analysis, using specific primers designed for detection only IL-15 payload (and not endogenous IL-15), showed high expression of IL-15 RNA, 4 day post-IL-15 engineered phage administration. 4 day post-phage administration, two tumors were phage-positive and both showed high expression of IL-15 payload RNA; mice 11, 79.

### Example 8

### Host Range Infectivity Assays

Plaque assay: Solid media assays for quantifying and isolating phage are based on plaque assays (S. T. Abedon et al., Methods in Molecular Biology 2009 (Clifton, N.J.), 501, 161-74), measuring the efficiency of plating (EOP) (E. Kutter, Methods in Molecular Biology 2009 (Clifton, N.J.), 501, 141-9) and spot testing (P. Hyman et al., Advances in Applied Microbiology (1st ed., Vol. 70, pp. 217-48) 2010. Elsevier Inc.).

A starter culture of 4 mL BHIS was inoculated with 50-100 colonies from a plate. This culture was incubated at 37° C. for 16 hours in an anaerobic environment. A volume of 200 μL of this culture was added to 5 mL of BHIS top agar (pre-molten 0.4% agar BHIS supplemented with 1 mM Ca²⁺, Mn²⁺ and Mg²⁺ ions), and the mixture was poured over a BHIS bottom agar plate (1.5% agar BHIS). The plates were left to solidify at room temperature and then, 5 μL of each of the samples containing phage (Titer of 10⁶ per ml), or media only (as controls) was dropped on the plate, and incubated anaerobically at 37° C. for 16 hours or more until plaques are visible for counting.

Tables 4 and 5 summarize the host infectivity of exemplary bacteriophages.

TABLE 4

| Bacteria | | | Fn1-1 | Fn2-58 | Fn5-1 | FN7-1 |
|---|---|---|---|---|---|---|
| name | Bacteria | Subspecies | ml/10⁶ | ml/10⁶ | ml/10⁶ | ml/10⁶ |
| FN1 | F. nucleatum | vincentii | + | − | − | − |
| FN2 | F. nucleatum | vincentii | − | + | − | − |
| FN11 | F. nucleatum | vincentii | − | − | − | − |
| FN14 | F. nucleatum | vincentii | − | − | − | − |
| FN3 | F. nucleatum | nucleatum | − | + | − | − |
| FNN | F. nucleatum | nucleatum | − | + | − | − |
| FN8 | F. nucleatum | nucleatum | − | + | − | + |
| FN12 | F. nucleatum | nucleatum | − | + | − | − |
| FN15 | F. nucleatum | nucleatum | − | NT | − | − |
| FN16 | F. nucleatum | nucleatum | − | NT | − | − |
| FNC1 | F. nucleatum | nucleatum | − | NT | − | + |
| FN4 | F. nucleatum | polymorphum | − | − | − | − |
| FNP | F. nucleatum | polymorphum | − | − | − | − |
| FNC2 | F. nucleatum | polymorphum | − | NT | − | + |
| FNC3 | F. nucleatum | polymorphum | | NT | − | − |
| FNC4 | F. nucleatum | polymorphum | | NT | − | − |
| FN5 | F. nucleatum | animalis | − | − | + | − |
| FN6 | F. nucleatum | animalis | − | − | − | − |
| FN7 | F. nucleatum | animalis | − | + | − | + |
| FNA | F. nucleatum | animalis | − | + | − | − |
| FN9 | F. nucleatum | animalis | − | + | − | − |
| FN13 | F. nucleatum | animalis | − | − | − | − |
| FN17 | F. nucleatum | animalis | − | NT | − | − |
| FN18 | F. nucleatum | animalis | − | NT | − | − |

TABLE 5

| Bacteria | | | Fn14-1 | Fn14-2 | Fn14-3 | Fn14-4 | Fn14-6 |
|---|---|---|---|---|---|---|---|
| name | Bacteria | Subspecies | ml/10⁶ | ml/10⁶ | ml/10⁶ | ml/10⁶ | ml/10⁶ |
| FN1 | F. nucleatum | vincentii | − | − | − | − | NT |
| FN2 | F. nucleatum | vincentii | − | + | − | − | − |
| FN11 | F. nucleatum | vincentii | − | − | − | − | NT |
| FN14 | F. nucleatum | vincentii | + | + | + | + | + |
| FN3 | F. nucleatum | nucleatum | − | − | − | − | NT |
| FNN | F. nucleatum | nucleatum | − | − | − | − | NT |
| FN8 | F. nucleatum | nucleatum | − | − | − | − | − |
| FN12 | F. nucleatum | nucleatum | − | − | − | − | NT |
| FN15 | F. nucleatum | nucleatum | NT | NT | NT | NT | NT |
| FN16 | F. nucleatum | nucleatum | NT | NT | NT | NT | NT |
| FNC1 | F. nucleatum | nucleatum | NT | NT | NT | NT | NT |
| FN4 | F. nucleatum | polymorphum | − | − | − | − | NT |
| FNP | F. nucleatum | polymorphum | − | − | − | − | NT |
| FNC2 | F. nucleatum | polymorphum | NT | NT | NT | NT | NT |
| FNC3 | F. nucleatum | polymorphum | NT | NT | NT | NT | NT |
| FNC4 | F. nucleatum | polymorphum | NT | NT | NT | NT | NT |
| FN5 | F. nucleatum | animalis | − | − | − | − | − |
| FN6 | F. nucleatum | animalis | NT | NT | NT | NT | NT |
| FN7 | F. nucleatum | animalis | − | − | − | − | − |
| FNA | F. nucleatum | animalis | − | − | − | − | NT |
| FN9 | F. nucleatum | animalis | − | − | − | − | − |
| FN13 | F. nucleatum | animalis | − | − | − | − | − |
| FN17 | F. nucleatum | animalis | NT | NT | NT | NT | NT |
| FN18 | F. nucleatum | animalis | NT | NT | NT | NT | NT |

“NT” represents pairs not included in the experiment.

### Example 9

### Natural Phage Accumulate in Bacteria Colonized Tumors

Materials and Methods

A mouse model of colorectal cancer was established in which tumors were successfully colonized by Fusobacterium nucleatum following IV-administration. Phage (native phage) was administered to the mouse by IV administration 3 days post bacteria administration and following 4 days, the tumors were extracted and the amount of bacteriophage was quantified, as described for Example 7.

Results

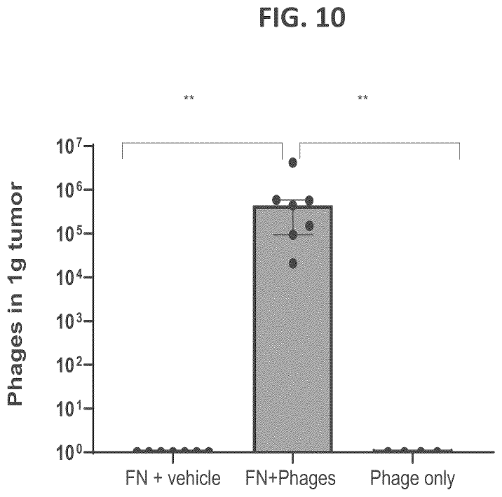

As illustrated in FIG. 10, following IV administration, phage home to and accumulate only in the tumors colonized with the bacteria. The high specificity and safety of phage make it a promising agent to deliver anti-tumor payloads to the tumor microenvironment.

### Example 10

### Decrease in F. nucleatum Load Following Natural Phage Treatment

Materials and Methods

A mouse model of colorectal cancer was established by subcutaneous injection of CT-26 cells to flank. Tumors were successfully colonized by Fusobacterium nucleatum following IV administration. Three days post bacteria administration, phage (Natural FN14-3 phage) was administered to the mouse by IV administration and following four days, the tumors were extracted and the amount of bacteriophage was quantified, as described for Example 7.

### Results

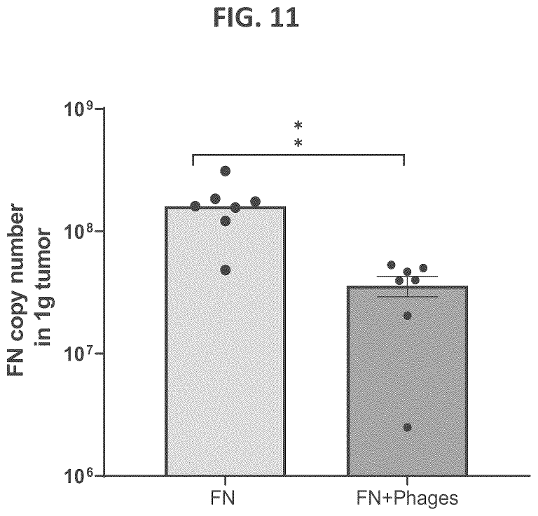

Native phage, administrated IV, led to a significant reduction in Fusobacterium nucleatum load in the colonized tumors, as illustrated in FIG. 11.

### Example 11

### In Vivo Reduction in Tumor Volume in Mice Treated with Phage

Material and Methods

The mouse model was set up as described for Example 9 and 10. 13 days post cancerous cell implantation, FN bacteria was IV administrated followed by multiple IV administrations of native phage. Tumor volume was subsequently measured using a caliper and was calculated using the formula (length×width2)/2.

### Results

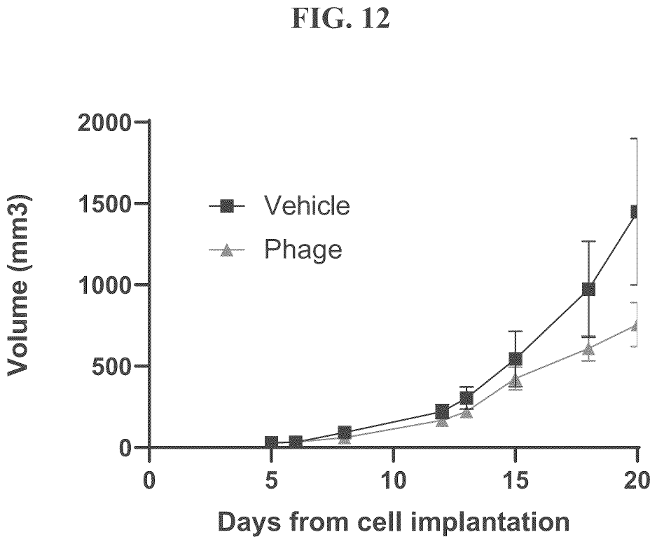

Tumor growth rate of mice following IV-administration of bacteria (day 13 post cancerous cell implantation) and IV multiple administrations of vehicle/phage treatment (days 16 &19). A significant reduction in tumor volume was observed 20 days post tumor cell implantation, as illustrated in FIG. 12. Reduction of tumoral burden by native phage or local expression of an anti-cancer/immune-stimulating payload to CRC by engineered phage may offer novel treatment approaches for patients with colorectal cancer.

Tumor growth inhibition (TGI) was calculated as 1−(mean tumor volume phage/mean tumor volume vehicle)*100. TGI was found to be 48%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

1. A method of treating a disease associated with a Fusobacterium nucleatum infection in a subject in need thereof, comprising administering to the subject a therapeutically effective amount of at least one isolated bacteriophage capable of infecting a Fusobacterium nucleatum bacterial species causing the infection,
wherein said at least one bacteriophage has a genomic nucleic acid sequence at least 85% identical to:
(i) the nucleic acid sequence as set forth in SEQ ID NOs: 1, 2, 3, 4, 5, 6, 7, 15, 16, 17, 18, 19 or 20, or
(ii) the genomic nucleic acid sequence of a bacteriophage deposited with the DSMZ under deposit number DSM 33306, DSM 33307, DSM 33582, DSM 33583, DSM 33639, DSM 33640, DSM 33641, DSM 33642 or DSM 33644,
thereby treating the disease associated with the Fusobacterium nucleatum infection.

2. (canceled)

3. The method of claim 1, wherein said disease is cancer.

4. (canceled)

5. The method of claim 3, wherein said cancer is selected from the group consisting of oral and esophageal cancer, colorectal cancer, pancreatic cancer, gastric cancer, gall bladder cancer, breast cancer, lung cancer, bladder cancer and melanoma.

6. The method of claim 3, wherein said administering comprises intratumoral administration.

7. The method of claim 1, further comprising identifying at least one strain of Fusobacterium nucleatum colonizing the subject prior to the administering.

8. (canceled)

9. The method of claim 1, wherein said at least one bacteriophage has a genomic nucleic acid sequence at least 85% identical to the nucleic acid sequence as set forth in SEQ ID NOs: 3, 4 or 5.

10. The method of claim 1, comprising administering to the subject at least two bacteriophages, wherein each of said at least two bacteriophages is capable of infecting a non-identical Fusobacterium nucleatum bacterial sub-species.

11. The method of claim 1, comprising administering to the subject two or more different bacteriophages, wherein said two or more different bacteriophages are capable of infecting different Fusobacterium nucleatum bacterial strains.

12. The method of claim 1, comprising administering to the subject at least two bacteriophages, wherein each of said at least two bacteriophages is capable of infecting an identical Fusobacterium nucleatum bacterial sub-species.

13. The method of claim 1, comprising administering to the subject two or more different bacteriophages, wherein at least two of the two or more bacteriophages are capable of infecting an identical Fusobacterium nucleatum bacterial sub-species.

14. The method of claim 1, wherein said at least one bacteriophage is genetically modified such that the genome thereof comprises a heterologous sequence.

15. The method of claim 14, wherein said heterologous sequence encodes a therapeutic agent or a diagnostic agent.

16. The method of claim 14, wherein said bacteriophage has a genomic nucleic acid sequence at least 85% identical to the nucleic acid sequence as set forth in SEQ ID NOs: 1, 2, 6 or 7, or a genomic nucleic acid sequence at least 85% identical to the genomic nucleic acid sequence of a bacteriophage deposited with the DSMZ under deposit number DSM 33306, DSM 33307, DSM 33639 or DSM 33644, and wherein said heterologous sequence renders said bacteriophage lytic.

17-23. (canceled)

24. A composition comprising two or more different isolated bacteriophages capable of infecting a Fusobacterium nucleatum bacterial strain, wherein at least one of said two or more different bacteriophages has a genomic nucleic acid sequence at least 85% identical to the nucleic acid sequence as set forth in SEQ ID NOs: 1, 2, 3, 4, 5, 6, 7, 15, 16, 17, 18, 19 or 20, or has a genomic nucleic acid sequence at least 85% identical to the genomic nucleic acid sequence of a bacteriophage deposited with the DSMZ under deposit number DSM 33306, DSM 33582, DSM 33307, DSM 33583, DSM 33639, DSM 33640, DSM 33641, DSM 33642 or DSM 33644.

25. (canceled)

26. The composition of claim 24, wherein each of said two or more bacteriophages is capable of infecting a non-identical Fusobacterium nucleatum bacterial sub-species.

27. The composition of claim 24, wherein each of said at least two bacteriophages is capable of infecting an identical Fusobacterium nucleatum bacterial sub-species.

28. The composition of claim 24, wherein at least two of said two or more bacteriophages is capable of infecting a non-identical Fusobacterium nucleatum bacterial sub-species.

29. The composition of claim 24, wherein at least two of said two or more bacteriophages are capable of infecting an identical Fusobacterium nucleatum bacterial sub-species.

30. The composition of claim 24, wherein said at least one bacteriophage is genetically modified such that the genome thereof comprises a heterologous sequence.

31-37. (canceled)

38. An isolated bacteriophage capable of infecting a Fusobacterium nucleatum bacterial species, wherein said bacteriophage has a genomic nucleic acid sequence at least 85% identical to the nucleic acid sequence as set forth in SEQ ID NOs: 1, 2, 3, 4, 5, 6, 7 15, 16, 17, 18, 19 or 20, or having a genomic nucleic acid sequence at least 85% identical to the genomic nucleic acid sequence of a bacteriophage deposited with the DSMZ under deposit number DSM 33307, DSM 33306, DSM 33582, DSM 33583, DSM 33639, DSM 33640, DSM 33641, DSM 33642 or DSM 33644.
